(19) United States
(12) Patent Application Publication
(10) Pub. No.: US 2008/0317666 A1
(43) Pub. Date: Dec. 25, 2008

(54) Colonic Delivery of Active Agents

(76) Inventors: Elias Fattal, Paris (FR); Antoine Andremont, Malakoff (FR); Patrick Couvreur, Villebon-sur-Yvette (FR); Sandrine Bourgeois, Lyon (FR)

Correspondence Address: David S. Bradin;Womble Carlyle Sandridge & Rice, P.O.Box 7037, Atlanta, GA, 30359-0037, US

(21) Appl. No.: 11/628,832

(22) PCT Filed: Feb. 9, 2006
(86) PCT No.: PCT/GB06/00448

§ 371 Date: Mar. 3, 2008

Related U.S. Application Data
(60) Provisional application No. 60/651,342, filed on Feb. 9, 2005.

(51) Int. Cl. A61K 9/00; A61K 49/00; A61K 51/12; A61P 1/00

(52) U.S. Cl. 424/1.11; 424/423; 424/9.1

(57) ABSTRACT

Drug delivery devices that are orally administered, and that release active ingredients in the colon, are disclosed. In one embodiment, the active ingredients are those that inactivate antibiotics, such as macrolides, quinolones and beta-lactam containing antibiotics. One example of a suitable active agent is an enzyme such as beta-lactamases. In another embodiment, the active agents are those that specifically treat colonic disorders, such as Chrohn's Disease, irritable bowel syndrome, ulcerative colitis, colorectal cancer or constipation. The drug delivery devices are in the form of beads of pectin, crosslinked with calcium and reticulated with polyethyleneimine. The high crosslink density of the polyethyleneimine is believed to stabilize the pectin beads for a sufficient amount of time such that a substantial amount of the active ingredients can be administered directly to the colon. Advantageously, the amount of polyethyleneimine is sufficient to allow a substantial portion of the pectin beads to pass through the gastrointestinal tract to the colon without releasing the active agent, and is also sufficient such that the pectin beads are sufficiently degraded in the colon to release an effective amount of the active agent.

### FIELD OF THE INVENTION

The present invention is in the area of oral drug delivery devices that administer active agents to the colon.

### BACKGROUND OF THE INVENTION

Drug delivery devices that specifically deliver active agents to the colon have been recognized as having important therapeutic advantages. A large number of colonic conditions could effectively be treated more efficaciously if the active ingredient is released locally. Examples of such colonic disorders include Crohn's disease, ulcerative colitis, colorectal cancer and constipation.

Colonic release can also benefit patients when, from a therapeutic point of view, a delay in absorption is necessary. Examples include the treatment of disorders such as nocturnal asthma or angor (Kinget R. et al. (1998), Colonic Drug Targeting, Journal of Drug Targeting, 6, 129).

Colonic release can also be used to administer therapeutically active polypeptides. Polypeptides are typically administered by injection, because they are degraded in the stomach. Because injection is painful, research efforts have focused on using the colon as a site of absorption for active polypeptides, including analgesics, contraceptives, vaccines, insulin, and the like. The absorption of polypeptides in the colon appears effectively better than in other sites in the digestive tract. This is particularly due to the relatively weak proteolytic activity in the small intestine and the absence of peptidasic activity associated with the membrane of the colonic epithelial cells.

During administration of antibiotics by mouth, they pass through the stomach and are then absorbed in the small intestine to diffuse in the whole organism and treat the infectious outbreak site for which they have been administered. All the same, a fraction of antibiotics ingested (whereof the importance varies with the characteristics of each type of antibiotics) is not absorbed and continues its progress to the colon before being eliminated in the stool. These residual antibiotics are reunited, in the large intestine, by a fraction of the antibiotics absorbed, but which are re-excreted in the digestive tract by means of biliary elimination. This fraction is of variable importance as a function of metabolism and ways of elimination of each antibiotic. Finally, for certain antibiotics, a fraction of the dose absorbed is eliminated directly via intestinal mucous in the lumen of the digestive tract. Thus, since the antibiotics had been administered orally or parenterally, a residual active fraction is generally found in the colon. This is true, to varying degrees, for the greater majority of families of antibiotics utilized in therapeutics, the sole notable exception being the family of amino-glycosides for which intestinal excretion is negligible. For other antibiotics, intestinal excretion of a residual antibiotic activity is going to have different consequences, all harmful. In effect, in the colon there is a complex and very dense bacterial ecosystem (several hundreds of different bacterial species; more than 10¹¹ bacteria per gram of colonic content) which is going to be affected by the arrival of active antibiotic residues. The following can be observed:

1. Imbalance in flora which would be the main cause of banal diarrhoea at time following taking antibiotics (Bartlett J. G. (2002) Clinical practice. Antibiotic associated diarrhoea, New England Journal of Medicine, 346, 334). Even though this diarrhoea is generally not serious and quickly ceases, either spontaneously, or on completion of treatment, it is all the same badly received by patients and adds to the discomfort of the base illness for which the antibiotic was prescribed;

2. perturbation of the functions of resistance to colonization by exogenic bacteria (or “barrier effect”) with possibilities of risk from infection, for example, alimentary salmonella intoxication (Holmberg S. D. et al. (1984) Drug resistant Salmonella from animals fed antimicrobials, New England Journal of Medicine, 311, 617);

3. selection of microorganisms resistant to the antibiotic. The latter can be of various types:

a) first they can be pathogenic bacteria such as for example, Clostridium difficile, a species capable of secreting toxins causing a form of colitis known as pseudomembranous (Bartlett J. G. (1997) Clostridium difficile infection: pathophysiology and diagnosis, Seminar in Gastrointestinal Disease, 8, 12);

b) they can also be microorganisms that are relatively weakly pathogenic, but whose multiplication can lead to an associated infection (vaginal Candidosis or Escherichia coli resistant cystitis).

c) they can finally be non-pathogenic commensal drug resistant bacteria whose multiplication and fecal elimination is going to increase dissemination in the environment. Now, these resistant commensal bacteria can constitute an important source of mechanisms of drug resistance for pathogenic species. This risk is currently considered seminal in terms of the disquieting character of the evolution towards drug multiresistance by numerous species pathogenic for humans.

Numerous strategies exploiting the diverse physiological parameters of the digestive tract have thus been envisioned with the aim of releasing active ingredients in the colon. These strategies have focused on drug delivery systems based on (1) using polymers that are sensitive to variations in pH, (2) time-dependent drug release forms, (3) prodrugs or polymers degradable by bacteria in the intestinal flora.

It would be advantageous to have additional drug delivery devices which can administer active agents to the colon. It would also be desirable to have drug delivery devices for reducing the quantity of residual antibiotics arriving at the colon after oral or parenteral antibiotic therapy. The present invention provides such drug delivery devices.

### SUMMARY OF THE INVENTION

Oral drug delivery devices that release active agents in the colon, are disclosed. In one embodiment, the active agents are those that inactivate antibiotics, such as macrolides, quinolones and beta-lactam containing antibiotics. One example of a suitable active agent is an enzyme such as beta-lactamases. In another embodiment, the active agents are those that specifically treat colonic disorders, such as ulcerative colitis, colorectal cancer, Chrohn's Disease, irritable bowel syndrome, and constipation. The active ingredients can be hydrosoluble or liposoluble. Depending on the active agent, the drug delivery devices can be used in therapeutics or in diagnostics.

The drug delivery devices are in the form of beads of pectin, crosslinked with calcium or other metal cations and reticulated with polyethyleneimine. The high crosslink density of the polyethyleneimine is believed to stabilize the pectin beads for a sufficient amount of time such that a substantial amount of the active ingredients can be administered directly to the colon.

The drug delivery devices include pectin beads in the form of a cationic salt, such as a calcium salt, including the active ingredient. The pectin is reticulated by polyethyleneimine. The molecular weight of the polyethyleneimine is between 10,000 and 100,000 Daltons, preferably between 20,000 and 50,000 Daltons. The pectin can be methylated or non-methylated, and amidated or non-amidated.

The pectin beads can be formulated into any type of drug delivery device suitable for oral delivery, including gelatine capsules, tablets and the like.

These drug delivery devices can be administered simultaneously or successively with other active ingredients. When they contain enzymes capable of inactivating antibiotics, they can be administered before, concurrently with, or after the preparations including the corresponding antibiotics. The manner in which the antibiotics are administered can vary, depending on the type of antibiotics, and can include oral or parenteral administration.

The drug delivery devices can be prepared using methods known to those of skill in the art, including by mixing the active agent in a pectin solution, crosslinking the pectin with a metal cation such as calcium to form pectin beads that encapsulate the active agent, and reticulating the beads with a solution of polyethyleneimine.

When the active agent is a beta-lactamase, the encapsulation yields are between 50 and 90% or 3-6 UI/beads of beta-lactamases, activity expressed in substrate benzylpenicillin, whether the pectin is amidated or not.

### BRIEF DESCRIPTION OF THE DRAWINGS

FIG. 1 illustrates the effect of reticulation with different concentrations of PEI (0.6; 0.7; 0.8; 0.9 and 1% (m/v)) on the disaggregation time of amidated pectin beads, placed in three different media: PBS, 0.01 M, pH at 7.4; intestinal medium at pH of 6.8±0.1 UPS XXIV; gastric medium at pH of 1.1 USP XXIV.

FIG. 2 illustrates the structure of the beads containing β-lactamases at the rate of 4.4 UI/bead and reticulated for 20 minutes at PEI of 1% and observed under electronic scanning microscopy.

FIG. 3 illustrates the release of β-lactamases in vitro from amidated reticulated beads of pectin prepared according to Example 1 with concentrations in PEI of 0.6 and 0.7% and containing around 5 UI/bead, placed in intestinal medium USP XXIV then in colonic medium (HEPES buffer pH 6+pectinolytic enzymes).

FIG. 4 illustrates the evolution of the β-lactamase activity in the stools of mice as a function of time, after oral administration of beads of pectin reticulated at PEI prepared according to Example 1 and containing 4.4 UI/bead.

FIG. 5 illustrates the structure of the beads containing β-lactamases at the rate of 4.4 UI/bead 30 minutes after in vivo administration. The beads are then in the stomach, with A and B representing the whole beads and C and D the cut beads.

FIG. 6 illustrates the structure of the beads containing β-lactamases at the rate of 4.4 UI/bead 2 hours after in vivo administration. The beads are then in the small intestine, with A and B representing the whole beads and C and D the cut beads.

FIG. 7 illustrates the structure of the beads containing β-lactamases at the rate of 4.4 UI/bead 4 hours after in vivo administration. The beads are then in the colon, with A and B representing the whole beads and C and D the cut beads.

FIG. 8 illustrates encapsulation, in beads of pectin, of free or complex plasmidic DNA with cationic lipids (Lipoplexe) or a cationic polymer (Polyplexe).

### DETAILED DESCRIPTION OF THE INVENTION

The drug delivery devices described herein will be better understood with reference to the following detailed description.

### I. Polyethyleneimine-Reticulated Pectin Beads

The polyethyleneimine-reticulated pectin beads of the present invention are formed from pectin, a polyvalent (i.e., divalent or trivalent) metal ion, and a cationic polymer, and encapsulate one or more active agents.

The pectin beads exhibit a stability in gastric medium greater than 10 hours and is likewise very good in intestinal medium USP XXIV, since it is greater than 7 hours, irrespective of the type of pectin used. In contrast, the duration of stability of non-reticulated pectin beads does not exceed 1 hour.

While not wishing to be bound to a particular theory, it is believed that the polyethyleneimine has a sufficient charge to weight ratio such that it can best stabilize the pectin beads. Other charged polymers, such as polylysine and chitosan, did not stabilize the pectin beads to nearly the same degree, and non-cationic polymers also did not sufficiently stabilize the pectin beads. Accordingly, it is believed that polyethyleneimine represents an optimum selection for a cationic polymer with which to reticulate, and thus stabilize, the pectin beads. Side-by-side comparisons of polyethyleneimine relative to other cationic polymers are provided in Example 8.

Pectin

Pectin is a polysaccharide isolated from the cellular walls of superior vegetables, used widely in the agricultural food industry (as a coagulant or thickener of jams, ice creams and the like) and pharmaceutics. It is polymolecular and polydisperse. Its composition varies according to the source, extraction conditions and environmental factors.

Pectins are principally composed of linear chains of beta-1,4-(D)-galacturonic acids, at times interspersed by units of rhamnose. The carboxylic groupings of galacturonic acids can be partially esterified to give methylated pectins. Two sorts of pectin are distinguished according to their degree of methylation (DM: number of methoxy group per 100 units of galacturonic acid):
highly methylated pectin (HM: high methoxy) whereof the degree of methylation varies between 50 and 80%. It is slightly soluble in water and forms gels in acidic medium (pH<3.6) or in the presence of sugars;
weakly methylated pectin (LM: low methoxy), with a degree of methylation varying from 25 to 50%. More soluble in water than pectin HM, it gives gels in the presence of divalent cations such as Ca²⁺ ions. In effect, Ca²⁺ ions form “bridges” between the carboxylated groups free of galacturonic acids. The network thus formed has been described by Grant et al. under the nom of <<egg-box model>>(Grant G. T. et al. (1973) Biological interactions between polysaccharides and divalent cations: the egg-box model, FEBS Letters, 32, 195).

There are also amidated pectins. Using treatment of pectin by ammonia certain methyl carboxylate groups (—COOCH₃) can be transformed into carboxamide groups (—CONH₂). This amidation confers novel properties on the pectins, especially better resistance to variations in pH. Amidated pectins tend to be more tolerant to the variations in pH, and have also been studied for elaboration of matricial tablets of colonic range (Wakerly Z. et al. (1997) Studies on amidated pectins as potential carriers in colonic drug delivery, Journal of Pharmacy and Pharmacology. 49, 622).

Pectin is degraded by enzymes originating from higher vegetables and various microorganisms (fungi, bacteria . . . ) among which bacteria of human colonic flora is found. The enzymes produced by the microflora are composed of a set of polysaccharidases, glycosidases and esterases.

Metal Cations

Any polyvalent (i.e., divalent, trivalent and the like) metal cation can be used to crosslink the pectin. Examples include calcium, zinc, aluminum, iron, and the like. Calcium is a preferred metal cation.

Polyethyleneimine

Polyethyleneimine is a strongly cationic polymer that binds to certain proteins, and is often used as a marker in immunology, to precipitate and purify enzymes and lipids. It is also known as aziridine polymer; epamine; epomine; ethylenimine polymer; montrek; PEI; and polymin(e). The molecular weight of the polyethyleneimine is between 10,000 and 100,000 Daltons, preferably between 20,000 and 50,000 Daltons.

The amount of polyethyleneimine used can be optimized, depending on the molecular weight and the type of pectin used. Advantageously, the optimal concentration for polyethyleneimine is that which provides reticulated pectin beads that are stable enough to survive in the gastrointestinal tract, yet unstable enough to be sufficiently degraded in the colon so as to release an effective amount of the active agent. Using the concentrations of components in the working examples, it was found that 0.8% (m/v) is the optimal concentration of polyethyleneimine to achieve these goals.

For example, when the pectin beads are prepared from a pectin solution at 4-10% (m/v), advantageously from 4 to 7% (m/v), and a solution of calcium chloride at 2-10% (m/v), a concentration of 0.8% (m/v) of polyethyleneimine (PEI) was optimal, but at 0.6% PEI (m/v), the beads were not enough sufficiently stable in the gastrointestinal tract to provide substantial colonic delivery, and at a concentration of 1% PEI (m/v), the beads were not sufficiently degraded in the colon to provide optimal delivery of the active agents. However, the beads at either the higher or lower concentration still released active agent, even though the release was non-optimal.

Those of skill in the art, using the teachings described herein, can readily optimize the amount of polyethyleneimine if there are variations in the concentration of pectin, the type of pectin, or the concentration or type of metal cation used, relative to that used in the working examples described herein.

Active Agents

The active agent can be an anti-infectious, for example antibiotics, anti-inflammatory compounds, anti-histamines, anti-cholinergics, antivirals, antimitotics, peptides, proteins, genes, anti-sense oligonucleotides, diagnostic agents and/or immunosuppressive agents or bacteria.

The active agent can be introduced into the drug delivery device as a powder, as a solution, or complexed with a solubilizing agent, such as a cyclodextrin.

Certain of the active agents described herein can be administered in the form of prodrugs. Prodrugs have been studied widely for colonic targeting of various active ingredients (such as non-steroidal and steroidal anti-inflammatories, and spasmolytics). These systems are based on the capacity of the enzymes produced by the colonic flora to degrade the prodrugs to release the active form of the active ingredient.

The prodrugs can be based on the action of bacterial azoreductases, so that the active agents are targeted to the colon with the drug delivery devices described herein, and the active agents are formed by reaction of the prodrug with a bacterial azoreductase, which provides a dual mechanism for ensuring that the drugs are administered to the colon. Representative chemistry for forming these prodrugs is described, for example, in Peppercorn M. A. et al. (1972) The role of intestinal bacteria in the metabolism of salicylazosulfapyridin, The Journal of Pharmacology and Experimental Therapeutics, 181, 555 and 64, 240.

Another approach consists of exploiting bacterial hydrolases such as glycosidases and polysaccharidases (Friend D. R. (1995) Glycoside prodrugs: novel pharmacotherapy for colonic diseases, S.T.P. Pharma Sciences, 5, 70; Friend D. R. et al. (1984) A colon-specific drug-delivery system based on drug glycosides and the glycosidases of colonic bacteria, Journal of Medicinal Chemistry, 27, 261; Friend D. R. et al. (1985) Drug glycosides: potential prodrugs for colon-specific drug delivery, Journal of Medicinal Chemistry, 28, 51; and Friend D. R. et al. (1992) Drug glycosides in oral colon-specific drug delivery, Journal of Controlled Release, 19, 109). Prodrugs have thus been developed by coupling, for example, sugar steroids (glucose, galactose, cellobiose, dextrane (international application WO 90/09168)), cyclodextrins Hirayama F. et al. (1996) In vitro evaluation of Biphenylyl Acetic Acid-beta-Cyclodextrin conjugates as colon-targeting prodrugs: drug release behavior in rat biological media, Journal of Pharmacy and Pharmacology, 48, 27).

a) Agents that Inactivate Antibiotics

In one embodiment, the active agent is an enzyme capable of inactivating antibiotics in the colon. When the antibiotic is a beta-lactam antibiotic, β-lactamases can be used. When the antibiotic is a macrolide or related substance, enzymes capable of inactivating macrolides and related substances, such as erythromycin esterase, can be used. The erythromycin esterase can be that disclosed by Andremont A. et al. ((1985) Plasmid mediated susceptibility to intestinal microbial antagonisms in Escherichia coli Infect. Immun. 49(3), 751), the contents of which are hereby incorporated by reference. When the antibiotic is a quinolone, the active agent can be one capable of inactivating quinolones. Representative agents include those disclosed by Chen Y et al. ((1997) Microbicidal models of soil metabolisms biotransformations of danofloxacin Journal of Industrial Microbiology and Biotechnology 19, 378).

b) Agents that Treat Colon Cancer

When the drug delivery devices are used to treat colon cancer, any type of antitumor agent can be used. The anti-tumor agents can be, for example, anti-proliferative agents, agents for DNA modification or repair, DNA synthesis inhibitors, DNA/RNA transcription regulators, enzyme activators, enzyme inhibitors, gene regulators, HSP-90 inhibitors, microtubule inhibitors, agents for phototherapy, and therapy adjuncts.

Representative antiproliferative agents include N-acetyl-D-sphingosine (C₂ ceramide), apigenin, berberine chloride, dichloromethylenediphosphonic acid disodium salt, loe-emodine, emodin, HA 14-1, N-hexanoyl-D-sphingosine (C₆ ceramide), 7b-hydroxycholesterol, 25-hydroxycholesterol, hyperforin, parthenolide, and rapamycin.

Representative agents for DNA modification and repair include aphidicolin, bleomycin sulfate, carboplatin, carmustine, chlorambucil, cyclophosphamide monohydrate, cyclophosphamide monohydrate ISOPAC®, cis-diammineplatinum(II) dichloride (Cisplatin), esculetin, melphalan, methoxyamine hydrochloride, mitomycin C, mitoxantrone dihydrochloride, oxaliplatin, and streptozocin.

Representative DNA synthesis inhibitors include (±)amethopterin (methotrexate), 3-amino-1,2,4-benzotriazine 1,4-dioxide, aminopterin, cytosine b-D-arabinofuranoside (Ara-C), cytosine b-D-arabinofuranoside (Ara-C) hydrochloride, 2-fluoroadenine-9-b-D-arabinofuranoside (Fludarabine des-phosphate; F-ara-A), 5-fluoro-5′-deoxyuridine, 5-fluorouracil, ganciclovir, hydroxyurea, 6-mercaptopurine, and 6-thioguanine.

Representative DNA/RNA transcription regulators include actinomycin D, daunorubicin hydrochloride, 5,6-dichlorobenzimidazole 1-b-D-ribofuranoside, doxorubicin hydrochloride, homoharringtonine, and idarubicin hydrochloride.

Representative enzyme activators and inhibitors include forskolin, DL-aminoglutethimide, apicidin, Bowman-Birk Inhibitor, butein, (S)-(+)-camptothecin, curcumin, (−)-deguelin, (−)-depudecin, doxycycline hyclate, etoposide, formestane, fostriecin sodium salt, hispidin, 2-imino-1-imidazolidineacetic acid (Cyclocreatine), oxamflatin, 4-phenylbutyric acid, roscovitine, sodium valproate, trichostatin A, tyrphostin AG 34, tyrphostin AG 879, urinary trypsin inhibitor fragment, valproic acid (2-propylpentanoic acid), and XK469.

Representative gene regulators include 5-aza-2′-deoxycytidine, 5-azacytidine, cholecalciferol (Vitamin D3), ciglitizone, cyproterone acetate, 15-deoxy-D^{12,14}-prostaglandin J₂, epitestosterone, flutamide, glycyrrhizic acid ammonium salt (glycyrrhizin), 4-hydroxytamoxifen, mifepristone, procainamide hydrochloride, raloxifene hydrochloride, all trans-retinal (vitamin A aldehyde), retinoic acid (vitamin A acid), 9-cis-retinoic acid, 13-cis-retinoic acid, retinoic acid p-hydroxyanilide, retinol (Vitamin A), tamoxifen, tamoxifen citrate salt, tetradecylthioacetic acid, and troglitazone.

Representative HSP-90 inhibitors include 17-(allylamino)-17-demethoxygeldanamycin and geldanamycin.

Representative microtubule inhibitors include colchicines, dolastatin 15, nocodazole, paclitaxel, podophyllotoxin, rhizoxin, vinblastine sulfate salt, vincristine sulfate salt, and vindesine sulfate salt and vinorelbine (Navelbine) ditartrate salt.

Representative agents for performing phototherapy include photoactive porphyrin rings, hypericin, 5-methoxypsoralen, 8-methoxypsoralen, psoralen and ursodeoxycholic acid.

Representative agents used as therapy adjuncts include amifostine, 4-amino-1,8-naphthalimide, brefeldin A, cimetidine, phosphomycin disodium salt, leuprolide (leuprorelin) acetate salt, luteinizing hormone-releasing hormone (LH-RH) acetate salt, lectin, papaverine hydrochloride, pifithrin-a, (−)-scopolamine hydrobromide, and thapsigargin.

The agents can also be anti-VEGF (vascular endothelial growth factor) agents, as such are known in the art. Several antibodies are currently in clinical trials or have been approved that function by inhibiting VEGF.

Some of the most commonly used antitumor agents currently in use or in clinical trials include paclitaxel, docetaxel, tamoxifen, vinorelbine, gemcitabine, cisplatin, etoposide, topotecan, irinotecan, anastrozole, rituximab, trastuzumab, fludarabine, cyclophosphamide, gentuzumab, carboplatin, interferon, and doxorubicin. The most commonly used anticancer agent is paclitaxel, which is used alone or in combination with other chemotherapy drugs such as: 5-FU, doxorubicin, vinorelbine, cytoxan, and cisplatin.

Combination therapy may be provided by combining two or more of the above compounds.

c) Agents that Treat Chrohn's Disease

There are several therapeutic approaches for treating Chrohn's Disease. Most people are first treated with drugs containing mesalamine, a substance that helps control inflammation. Sulfasalazine is the most commonly used of these drugs. Patients who do not benefit from it or who cannot tolerate it may be put on other mesalamine-containing drugs, generally known as 5-ASA agents, such as Asacol, Dipentum, or Pentasa. Corticosteroids are often administered to control inflammation.

Immunosuppressive agents are also used to treat Crohn's disease. Most commonly prescribed are 6-mercaptopurine and a related drug, azathioprine. Immunosuppressive agents work by blocking the immune reaction that contributes to inflammation.

Patients can be treated with combinations of these agents, for example, combinations of corticosteroids and immunosuppressive drugs.

The U.S. Food and Drug Administration has approved the drug infliximab (brand name, Remicade) for the treatment of moderate to severe Crohn's disease that does not respond to standard therapies (mesalamine substances, corticosteroids, immunosuppressive agents) and for the treatment of open, draining fistulas. Infliximab is an anti-tumor necrosis factor (TNF) substance. This and other anti-TNF agents can be used to remove TNF from the colon, thereby preventing inflammation, without the side effects that might result if TNF were removed from the blood stream outside of the colon.

Antidiarrheal agents are often also administered, including diphenoxylate, loperamide, and codeine.

d) Agents that Treat Ulcerative Colitis

The agents that are used to treat ulcerative colitis overlap with those used to treat Chrohn's Disease. Examples include aminosalicylates, drugs that contain 5-aminosalicyclic acid (5-ASA), to help control inflammation, such as sulfasalazine, olsalazine, mesalamine, and balsalazide. They also include corticosteroids such as prednisone and hydrocortisone, immunomodulators such as azathioprine and 6-mercapto-purine (6-MP). Cyclosporine A may be used with 6-MP or azathioprine to treat active, severe ulcerative colitis. TNF alpha, the thiazoldinediones or glitazones, including rosiglitazone, can also be used.

e) Agents that Treat Constipation/Irritable Bowel Syndrome

Constipation, such as that associated with irritable bowel syndrome, is often treated using stimulant laxatives, osmotic laxatives such as Lactulose and MiraLax, stool softeners (such as mineral oil or Colace), bulking agents (such as Metamucil or bran). Agents such as Zelnorm (also called tegaserod) can be used to treat IBS with constipation. Additionally, anticholinergic medications such as Bentyl® and Levsin® have been found to be helpful in alleviating the bowel spasms of IBS.

f) Protein and Peptide Drugs

The drug delivery devices can be used to orally administer proteins and peptides that might otherwise be degraded if orally administered, and which might otherwise have to be administered intramuscularly or intravenously.

Examples of protein and peptide drugs useful in the present invention include:

Adrenocorticotropic hormone (ACTH) peptides including, but not limited to, ACTH, human; ACTH 1-10; ACTH 1-13, human; ACTH 1-16, human; ACTH 1-17; ACTH 1-24, human; ACTH 4-10; ACTH 4-11; ACTH 6-24; ACTH 7-38, human; ACTH 18-39, human; ACTH, rat; ACTH 12-39, rat; beta-cell tropin (ACTH 22-39); biotinyl-ACTH 1-24, human; biotinyl-ACTH 7-38, human; corticostatin, human; corticostatin, rabbit; [Met(02)⁴, DLys⁸, Phe⁹] ACTH 4-9, human; [Met(0)⁴,DLys⁸, Phe⁹] ACTH 4-9, human; N-acetyl, ACTH 1-17, human; and ebiratide.

Adrenomedullin peptides including, but not limited to, adrenomedullin, adrenomedullin 1-52, human; adrenomedullin 1-12, human; adrenomedullin 13-52, human; adrenomedullin 22-52, human; pro-adrenomedullin 45-92, human; pro-adrenomedullin 153-185, human; adrenomedullin 1-52, porcine; pro-adrenomedullin (N-20), porcine; adrenomedullin 1-50, rat; adrenomedullin 11-50, rat; and proAM-N20 (proadrenomedullin N-terminal 20 peptide), rat.

Allatostatin peptides including, but not limited to, allatostatin I; allatostatin II; allatostatin III; and allatostatin IV.

Amylin peptides including, but not limited to, acetyl-amylin 8-37, human; acetylated amylin 8-37, rat; AC187 amylin antagonist; AC253 amylin antagonist; AC625 amylin antagonist; amylin 8-37, human; amylin (IAPP), cat; amylin (insulinoma or islet amyloid polypeptide(IAPP)); amylin amide, human; amylin 1-13 (diabetes-associated peptide 1-13), human; amylin 20-29 (IAPP 20-29), human; AC625 amylin antagonist; amylin 8-37, human; amylin (IAPP), cat; amylin, rat; amylin 8-37, rat; biotinyl-amylin, rat; and biotinyl-amylin amide, human.

Amyloid beta-protein fragment peptides including, but not limited to, Alzheimer's disease beta-protein 12-28 (SP17); amyloid beta-protein 25-35; amyloid beta/A4-protein precursor 328-332; amyloid beta/A4 protein precursor (APP) 319-335; amyloid beta-protein 1-43; amyloid beta-protein 1-42; amyloid beta-protein 1-40; amyloid beta-protein 10-20; amyloid beta-protein 22-35; Alzheimer's disease beta-protein (SP28); beta-amyloid peptide 1-42, rat; beta-amyloid peptide 1-40, rat; beta-amyloid 1-11; beta-amyloid 31-35; beta-amyloid 32-35; beta-amyloid 35-25; beta-amyloid/A4 protein precursor 96-110; beta-amyloid precursor protein 657-676; beta-amyloid 1-38; [Gln¹¹]-Alzheimer's disease beta-protein; [Gln¹¹]-beta-amyloid 1-40; [Gln²²]-beta-amyloid 6-40; non-A beta component of Alzheimer's disease amyloid (NAC); P3, (A beta 17-40) Alzheimer's disease amyloid β-peptide; and SAP (serum amyloid P component) 194-204.

Angiotensin peptides including, but not limited to, A-779; Ala-Pro-Gly-angiotensin II; [Ile³,Val⁵]-angiotensin II; angiotensin III antipeptide; angiogenin fragment 108-122; angiogenin fragment 108-123; angiotensin I converting enzyme inhibitor; angiotensin I, human; angiotensin I converting enzyme substrate; angiotensin 11-7, human; angiopeptin; angiotensin II, human; angiotensin II antipeptide; angiotensin II 1-4, human; angiotensin II 3-8, human; angiotensin II 4-8, human; angiotensin II 5-8, human; angiotensin III ([Des-Asp¹]-angiotensin II), human; angiotensin III inhibitor ([Ile⁷]-angiotensin III); angiotensin-converting enzyme inhibitor (Neothunnus macropterus); [Asn¹, Val⁵]-angiotensin I, goosefish; [Asn¹, Val⁵, Asn⁹]-angiotensin I, salmon; [Asn¹, Val⁵, Gly⁹]-angiotensin I, eel; [Asn¹, Val⁵]-angiotensin I 1-7, eel, goosefish, salmon; [Asn¹, Val⁵]-angiotensin II; biotinyl-angiotensin I, human; biotinyl-angiotensin II, human; biotinyl-Ala-Ala-Ala-angiotensin II; [Des-Asp¹]-angiotensin I, human; [p-aminophenylalanine⁶]-angiotensin II; renin substrate (angiotensinogen 1-13), human; preangiotensinogen 1-14 (renin substrate tetradecapeptide), human; renin substrate tetradecapeptide (angiotensinogen 1-14), porcine; [Sar¹]-angiotensin II, [Sar¹]-angiotensin II 1-7 amide; [Sar¹, Ala⁸]-angiotensin II; [Sar¹, Ile⁸]-angiotensin II; [Sar¹, Thr⁸]-angiotensin II; [Sar¹, Tyr(Me)⁴]-angiotensin II (Sarmesin); [Sar¹, Val⁵, Ala⁸]-angiotensin II; [Sar¹, Ile⁷]-angiotensin III; synthetic tetradecapeptide renin substrate (No. 2); [Val⁴]-angiotensin III; [Val⁵]-angiotensin II; [Val⁵]-angiotensin I, human; [Val⁵]-angiotensin I; [Val⁵, Asn⁹]-angiotensin I, bullfrog; and [Val⁵, Ser⁹]-angiotensin I, fowl.

Antibiotic peptides including, but not limited to, Ac-SQNY; bactenecin, bovine; CAP 37 (20-44); carbormethoxycarbonyl-DPro-DPhe-OBzl; CD36 peptide P 139-155; CD36 peptide P 93-110; cecropin A-melittin hybrid peptide [CA(1-7)M(2-9)NH2]; cecropin B, free acid; CYS(Bzl)84 CD fragment 81-92; defensin (human) HNP-2; dermaseptin; immunostimulating peptide, human; lactoferricin, bovine (BLFC); and magainin spacer.

Antigenic polypeptides, which can elicit an enhanced immune response, enhance an immune response and or cause an immunizingly effective response to diseases and/or disease causing agents including, but not limited to, adenoviruses; anthrax; Bordetella pertussus; botulism; bovine rhinotracheitis; Branhamella catarrhalis; canine hepatitis; canine distemper; Chlamydiae; cholera; coccidiomycosis; cowpox; cytomegalovirus; Dengue fever; dengue toxoplasmosis; diphtheria; encephalitis; enterotoxigenic E. coli; Epstein Barr virus; equine encephalitis; equine infectious anemia; equine influenza; equine pneumonia; equine rhinovirus; Escherichia coli; feline leukemia; flavivirus; globulin; haemophilus influenza type b; Haemophilus influenzae; Haemophilus pertussis; Helicobacter pylon; hemophilus; hepatitis; hepatitis A; hepatitis B; Hepatitis C; herpes viruses; HIV; HIV-1 viruses; HIV-2 viruses; HTLV; influenza; Japanese encephalitis; Klebsiellae species; Legionella pneumophila; leishmania; leprosy; lyme disease; malaria immunogen; measles; meningitis; meningococcal; Meningococcal polysaccharide group A; Meningococcal polysaccharide group C; mumps; mumps virus; mycobacteria; Mycobacterium tuberculosis; Neisseria; Neisseria gonorrhea; Neisseria meningitidis; ovine blue tongue; ovine encephalitis; papilloma; parainfluenza; paramyxoviruses; Pertussis; plague; pneumococcus; Pneumocystis carinii; pneumonia; poliovirus; proteus species; Pseudomonas aeruginosa; rabies; respiratory syncytial virus; rotavirus; rubella; salmonellae; schistosomiasis; shigellae; simian immunodeficiency virus; smallpox; Staphylococcus aureus; Staphylococcus species; Streptococcus pneumoniae; Streptococcus pyogenes; Streptococcus species; swine influenza; tetanus; Treponema pallidum; typhoid; vaccinia; varicella-zoster virus; and vibrio cholerae.

Anti-microbial peptides including, but not limited to, buforin I; buforin II; cecropin A; cecropin B; cecropin P1, porcine; gaegurin 2 (Rana rugosa); gaegurin 5 (Rana rugosa); indolicidin; protegrin-(PG)-I; magainin 1; and magainin 2; and T-22 [Tyr^{5,12}, Lys⁷]-poly-phemusin II peptide.

Apoptosis related peptides including, but not limited to, Alzheimer's disease beta-protein (SP28); calpain inhibitor peptide; capsase-1 inhibitor V; capsase-3, substrate IV; caspase-1 inhibitor I, cell-permeable; caspase-1 inhibitor VI; caspase-3 substrate III, fluorogenic; caspase-1 substrate V, fluorogenic; caspase-3 inhibitor I, cell-permeable; caspase-6 ICE inhibitor III; [Des-Ac, biotin]-ICE inhibitor III; IL-1 B converting enzyme (ICE) inhibitor II; IL-1 B converting enzyme (ICE) substrate IV; MDL 28170; and MG-132.

Atrial natriuretic peptides including, but not limited to, alpha-ANP (alpha-chANP), chicken; anantin; ANP 1-11, rat; ANP 8-30, frog; ANP 11-30, frog; ANP-21 (fANP-21), frog; ANP-24 (fANP-24), frog; ANP-30, frog; ANP fragment 5-28, human, canine; ANP-7-23, human; ANP fragment 7-28, human, canine; alpha-atrial natriuretic polypeptide 1-28, human, canine; A71915, rat; atrial natriuretic factor 8-33, rat; atrial natriuretic polypeptide 3-28, human; atrial natriuretic polypeptide 4-28, human, canine; atrial natriuretic polypeptide 5-27; human; atrial natriuretic aeptide (ANP), eel; atriopeptin I, rat, rabbit, mouse; atriopeptin II, rat, rabbit, mouse; atriopeptin III, rat, rabbit, mouse; atrial natriuretic factor (rANF), rat, auriculin A (rat ANF 126-149); auriculin B (rat ANF 126-150); beta-ANP (1-28, dimer, antiparallel); beta-rANF 17-48; biotinyl-alpha-ANP 1-28, human, canine; biotinyl-atrial natriuretic factor (biotinyl-rANF), rat; cardiodilatin 1-16, human; C-ANF 4-23, rat; Des-[Cys¹⁰⁵, Cys¹²¹]-atrial natriuretic factor 104-126, rat; [Met(O)¹²] ANP 1-28, human; [Mpr⁷,DAla⁹]ANP 7-28, amide, rat; prepro-ANF 104-116, human; prepro-ANF 26-55 (proANF 1-30), human; prepro-ANF 56-92 (proANF 31-67), human; prepro-ANF 104-123, human; [Tyr⁰]-atriopeptin I, rat, rabbit, mouse; [Tyr⁰]-atriopeptin II, rat, rabbit, mouse; [Tyr⁰]-prepro ANF 104-123, human; urodilatin (CDD/ANP 95-126); ventricular natriuretic peptide (VNP), eel; and ventricular natriuretic peptide (VNP), rainbow trout.

Bag cell peptides including, but not limited to, alpha bag cell peptide; alpha-bag cell peptide 1-9; alpha-bag cell peptide 1-8; alpha-bag cell peptide 1-7; beta-bag cell factor; and gamma-bag cell factor.

Bombesin peptides including, but not limited to, alpha-s1 casein 101-123 (bovine milk); biotinyl-bombesin; bombesin 8-14; bombesin; [Leu¹³-psi (CH2NH)Leu¹⁴]-bombesin; [D-Phe⁶, Des-Met¹⁴]-bombesin 6-14 ethylamide; [DPhe¹²] bombesin; [DPhe¹²,Leu¹⁴]-bombesin; [Tyr⁴]-bombesin; and [Tyr⁴,DPhe¹²]-bombesin.

Bone GLA peptides (BGP) including, but not limited to, bone GLA protein; bone GLA protein 45-49; [Glu¹⁷, Gla^{21,24}]-osteocalcin 1-49, human; myclopeptide-2 (MP-2); osteocalcin 1-49 human; osteocalcin 37-49, human; and [Tyr³⁸, Phe^{42,46}] bone GLA protein 38-49, human.

Bradykinin peptides including, but not limited to, [Ala^{2,6}, des-Pro³]-bradykinin; bradykinin; bradykinin (Bowfin. Gar); bradykinin potentiating peptide; bradykinin 1-3; bradykinin 1-5; bradykinin 1-6; bradykinin 1-7; bradykinin 2-7; bradykinin 2-9; [DPhe⁷] bradykinin; [Des-Arg⁹]-bradykinin; [Des-Arg¹⁰]-Lys-bradykinin ([Des-Arg¹⁰]-kallidin); [D-N-Me-Phe⁷]-bradykinin; [Des-Arg⁹, Leu⁸]-bradykinin; Lys-bradykinin (kallidin); Lys-[Des-Arg⁹,Leu⁸]-bradykinin ([Des-Arg¹⁰,Leu⁹]-kallidin); [Lys⁰-Hyp³]-bradykinin; ovokinin; [Lys⁰, Ala³]-bradykinin; Met-Lys-bradykinin; peptide K12 bradykinin potentiating peptide; [(pCl)Phe^{5,8}]-bradykinin; T-kinin (Ile-Ser-bradykinin); [Thi^{5,8}, D-Phe⁷]-bradykinin; [Tyr⁰]-bradykinin; [Tyr⁵]-bradykinin; [Tyr⁸]-bradykinin; and kallikrein.

Brain natriuretic peptides (BNP) including, but not limited to, BNP 32, canine; BNP-like Peptide, eel; BNP-32, human; BNP-45, mouse; BNP-26, porcine; BNP-32, porcine; biotinyl-BNP-32, porcine; BNP-32, rat; biotinyl-BNP-32, rat; BNP-45 (BNP 51-95, 5K cardiac natriuretic peptide), rat; and [Tyr⁰]-BNP 1-32, human.

C-peptides including, but not limited to, C-peptide; and [Tyr⁰]-C-peptide, human.

C-type natriuretic peptides (CNP) including, but not limited to, C-type natriuretic peptide, chicken; C-type natriuretic peptide-22 (CNP-22), porcine, rat, human; C-type natriuretic peptide-53 (CNP-53), human; C-type natriuretic peptide-53 (CNP-53), porcine, rat; C-type natriuretic peptide-53 (porcine, rat) 1-29 (CNP-53 1-29); prepro-CNP 1-27, rat; prepro-CNP 30-50, porcine, rat; vasonatrin peptide (VNP); and [Tyr⁰]-C-type natriuretic peptide-22 ([Tyr⁰]-CNP-22).

Calcitonin peptides including, but not limited to, biotinyl-calcitonin, human; biotinyl-calcitonin, rat; biotinyl-calcitonin, salmon; calcitonin, chicken; calcitonin, eel; calcitonin, human; calcitonin, porcine; calcitonin, rat; calcitonin, salmon; calcitonin 1-7, human; calcitonin 8-32, salmon; katacalcin (PDN-21) (C-procalcitonin); and N-proCT (amino-terminal procalcitonin cleavage peptide), human.

Calcitonin gene related peptides (CGRP) including, but not limited to, acetyl-alpha-CGRP 19-37, human; alpha-CGRP 19-37, human; alpha-CGRP 23-37, human; biotinyl-CGRP, human; biotinyl-CGRP II, human; biotinyl-CGRP, rat; beta-CGRP, rat; biotinyl-beta-CGRP, rat; CGRP, rat; CGRP, human; calcitonin C-terminal adjacent peptide; CGRP 1-19, human; CGRP 20-37, human; CGRP 8-37, human; CGRP II, human; CGRP, rat; CGRP 8-37, rat; CGRP 29-37, rat; CGRP 30-37, rat; CGRP 31-37, rat; CGRP 32-37, rat; CGRP 33-37, rat; CGRP 31-37, rat; ([Cys(Acm)^{2,7}]-CGRP; elcatonin; [Tyr⁰]-CGRP, human; [Tyr⁰]-CGRP II, human; [Tyr⁰]-CGRP 28-37, rat; [Tyr⁰]-CGRP, rat; and [Tyr²²]-CGRP 22-37, rat.

CART peptides including, but not limited to, CART, human; CART 55-102, human; CART, rat; and CART 55-102, rat.

Casomorphin peptides including, but not limited to, beta-casomorphin, human; beta-casomorphin 1-3; beta-casomorphin 1-3, amide; beta-casomorphin, bovine; beta-casomorphin 1-4, bovine; beta-casomorphin 1-5, bovine; beta-casomorphin 1-5, amide, bovine; beta-casomorphin 1-6, bovine; [DAla²]-beta-casomorphin 1-3, amide, bovine; [DAla²,Hyp⁴,Tyr⁵]-beta-casomorphin 1-5 amide; [DAla²,DPro⁴,Tyr⁵]-beta-casomorphin 1-5, amide; [DAla²,Tyr⁵]-beta-casomorphin 1-5, amide, bovine; [DAla^{2,4},Tyr⁵]-beta-casomorphin 1-5, amide, bovine; [DAla², (pCl)Phe³]-beta-casomorphin, amide, bovine; [DAla²]-beta-casomorphin 1-4, amide, bovine; [DAla²]-beta-casomorphin 1-5, bovine; [DAla²]-beta-casomorphin 1-5, amide, bovine; [DAla²,Met⁵]-beta-casomorphin 1-5, bovine; [DPro²]-beta-casomorphin 1-5, amide, bovine; [DAla²]-beta-casomorphin 1-6, bovine; [DPro²]-beta-casomorphin 1-4, amide; [Des-Tyr¹]-beta-casomorphin, bovine; [DAla^{2,4},Tyr⁵]-beta-casomorphin 1-5, amide, bovine; [DAla², (pCl)Phe³]-beta-casomorphin, amide, bovine; [DAla²]-beta-casomorphin 1-4, amide, bovine; [DAla²]-beta-casomorphin 1-5, bovine; [DAla²]-beta-casomorphin 1-5, amide, bovine; [DAla²,Met⁵]-beta-casomorphin 1-5, bovine; [DPro²]-beta-casomorphin 1-5, amide, bovine; [DAla²]-beta-casomorphin 1-6, bovine; [DPro²]-beta-casomorphin 1-4, amide; [Des-Tyr¹]-beta-casomorphin, bovine; and [Val³]-beta-casomorphin 1-4, amide, bovine.

Chemotactic peptides including, but not limited to, defensin 1 (human) HNP-1 (human neutrophil peptide-1); and N-formyl-Met-Leu-Phe.

Cholecystokinin (CCK) peptides including, but not limited to, caerulein; cholecystokinin; cholecystokinin-pancreozymin; CCK-33, human; cholecystokinin octapeptide 1-4 (non-sulfated) (CCK 26-29, unsulfated); cholecystokinin octapeptide (CCK 26-33); cholecystokinin octapeptide (non-sulfated) (CCK 26-33, unsulfated); cholecystokinin heptapeptide (CCK 27-33); cholecystokinin tetrapeptide (CCK 30-33); CCK-33, porcine; CR 1 409, cholecystokinin antagonist; CCK flanking peptide (unsulfated); N-acetyl cholecystokinin, CCK 26-30, sulfated; N-acetyl cholecystokinin, CCK 26-31, sulfated; N-acetyl cholecystokinin, CCK 26-31, non-sulfated; prepro CCK fragment V-9-M; and proglumide.

Colony-stimulating factor peptides including, but not limited to, colony-stimulating factor (CSF); GMCSF; MCSF; and G-CSF.

Corticortropin releasing factor (CRF) peptides including, but not limited to, astressin; alpha-helical CRF 12-41; biotinyl-CRF, ovine; biotinyl-CRF, human, rat; CRF, bovine; CRF, human, rat; CRF, ovine; CRF, porcine; [Cys²¹]-CRF, human, rat; CRF antagonist (alpha-helical CRF 9-41); CRF 6-33, human, rat; [DPro⁵]-CRF, human, rat; [D-Phe¹², Nle^{21,38}]-CRF 12-41, human, rat; eosinophilotactic peptide; [Met(0)²¹]-CRF, ovine; [Nle²¹,Tyr³²]-CRF, ovine; prepro CRF 125-151, human; sauvagine, frog; [Tyr⁰]-CRF, human, rat; [Tyr⁰]-CRF, ovine; [Tyr⁰]-CRF 34-41, ovine; [Tyr⁰]-urocortin; urocortin amide, human; urocortin, rat; urotensin I (Catostomus commersoni); urotensin II; and urotensin II (Rana ridibunda).

Cortistatin peptides including, but not limited to, cortistatin 29; cortistatin 29 (1-13); [Tyr⁰]-cortistatin 29; pro-cortistatin 28-47; and pro-cortistatin 51-81.

Cytokine peptides including, but not limited to, tumor necrosis factor; and tumor necrosis factor-β (TNF-β).

Dermorphin peptides including, but not limited to, dermorphin and dermorphin analog 1-4.

Dynorphin peptides including, but not limited to, big dynorphin (prodynorphin 209-240), porcine; biotinyl-dynorphin A (biotinyl-prodynorphin 209-225); [DAla², DArg⁶]-dynorphin A 1-13, porcine; [D-Ala²]-dynorphin A, porcine; [D-Ala²]-dynorphin A amide, porcine; [D-Ala²]-dynorphin A 1-13, amide, porcine; [D-Ala²]-dynorphin A 1-9, porcine; [DArg⁶]-dynorphin A 1-13, porcine; [DArg⁸]-dynorphin A 1-13, porcine; [Des-Tyr¹]-dynorphin A 1-8; [D-Pro¹⁰]-dynorphin A 1-11, porcine; dynorphin A amide, porcine; dynorphin A 1-6, porcine; dynorphin A 1-7, porcine; dynorphin A 1-8, porcine; dynorphin A 1-9, porcine; dynorphin A 1-10, porcine; dynorphin A 1-10 amide, porcine; dynorphin A 1-11, porcine; dynorphin A 1-12, porcine; dynorphin A-13, porcine; dynorphin A 1-13 amide, porcine; DAKLI (dynorphin A-analogue kappa ligand); DAKLI-biotin ([Arg^{11,13}]-dynorphin A (1-13)-Gly-NH(CH2)5NH-biotin); dynorphin A 2-17, porcine; dynorphin 2-17, amide, porcine; dynorphin A 2-12, porcine; dynorphin A 3-17, amide, porcine; dynorphin A 3-8, porcine; dynorphin A 3-13, porcine; dynorphin A 3-17, porcine; dynorphin A 7-17, porcine; dynorphin A 8-17, porcine; dynorphin A 6-17, porcine; dynorphin A 13-17, porcine; dynorphin A (prodynorphin 209-225), porcine; dynorphin B 1-9; [MeTyr¹, MeArg⁷, D-Leu⁸]-dynorphin 1-8 ethyl amide; [(nMe)Tyr¹] dynorphin A 1-13, amide, porcine; [Phe⁷]-dynorphin A 1-7, porcine; [Phe⁷]-dynorphin A 1-7, amide, porcine; and prodynorphin 228-256 (dynorphin B 29) (leumorphin), porcine.

Endorphin peptides including, but not limited to, alpha-neo-endorphin, porcine; beta-neo-endorphin; Ac-beta-endorphin, camel, bovine, ovine; Ac-beta-endorphin 1-27, camel, bovine, ovine; Ac-beta-endorphin, human; Ac-beta-endorphin 1-26, human; Ac-beta-endorphin 1-27, human; Ac-gamma-endorphin (Ac-beta-lipotropin 61-77); acetyl-alpha-endorphin; alpha-endorphin (beta-lipotropin 61-76); alpha-neo-endorphin analog; alpha-neo-endorphin 1-7; [Arg⁸]-alpha-neo-endorphin 1-8; beta-endorphin (beta-lipotropin 61-91), camel, bovine, ovine; beta-endorphin 1-27, camel, bovine, ovine; beta-endorphin, equine; beta-endorphin (beta-lipotropin 61-91), human; beta-endorphin (1-5)+(16-31), human; beta-endorphin 1-26, human; beta-endorphin 1-27, human; beta-endorphin 6-31, human; beta-endorphin 18-31, human; beta-endorphin, porcine; beta-endorphin, rat; beta-lipotropin 1-10, porcine; beta-lipotropin 60-65; beta-lipotropin 61-64; beta-lipotropin 61-69; beta-lipotropin 88-91; biotinyl-beta-endorphin (biotinyl-beta-lipotropin 61-91); biocytin-beta-endorphin, human; gamma-endorphin (beta-lipotropin 61-77); [DAla²]-alpha-neo-endorphin 1-2, amide; [DAla²]-beta-lipotropin 61-69; [DAla²]-gamma-endorphin; [Des-Tyr¹]-beta-endorphin, human; [Des-Tyr¹]-gamma-endorphin (beta-lipotropin 62-77); [Leu⁵]-beta-endorphin, camel, bovine, ovine; [Met⁵, Lys⁶]-alpha-neo-endorphin 1-6; [Met⁵, Lys^{6,7}]-alpha-neo-endorphin 1-7; and [Met⁵, Lys⁶, Arg⁷]-alpha-neo-endorphin 1-7.

Endothelin peptides including, but not limited to, endothelin-1 (ET-1); endothelin-1[Biotin-Lys⁹]; endothelin-1 (1-15), human; endothelin-1 (1-15), amide, human; Ac-endothelin-1 (16-21), human; Ac-[DTrp¹⁶]-endothelin-1 (16-21), human; [Ala^{3,11}]-endothelin-1; [Dprl, Asp¹⁵]-endothelin-1; [Ala²]-endothelin-3, human; [Ala¹⁸]-endothelin-1, human; [Asn¹⁸]-endothelin-1, human; [Res-701-1]-endothelin B receptor antagonist; Suc-[Glu⁹, Ala^{11,15}]-endothelin-1 (8-21), IRL-1620; endothelin-C-terminal hexapeptide; [D-Val²²]-big endothelin-1 (16-38), human; endothelin-2 (ET-2), human, canine; endothelin-3 (ET-3), human, rat, porcine, rabbit; biotinyl-endothelin-3 (biotinyl-ET-3); prepro-endothelin-1 (94-109), porcine; BQ-518; BQ-610; BQ-788; endothelium-dependent relaxation antagonist; FR139317; IRL-1038; JKC-30 1; JKC-302; PD-145065; PD 142893; sarafotoxin S6a (atractaspis engaddensis); sarafotoxin S6b (atractaspis engaddensis); sarafotoxin S6c (atractaspis engaddensis); [Lys⁴]-sarafotoxin S6c; sarafotoxin S6d; big endothelin-1, human; biotinyl-big endothelin-1, human; big endothelin-1 (1-39), porcine; big endothelin-3 (22-41), amide, human; big endothelin-1 (22-39), rat; big endothelin-1 (1-39), bovine; big endothelin-1 (22-39), bovine; big endothelin-1 (19-38), human; big endothelin-1 (22-38), human; big endothelin-2, human; big endothelin-2 (22-37), human; big endothelin-3, human; big endothelin-1, porcine; big endothelin-1 (22-39) (prepro-endothelin-1 (74-91)); big endothelin-1, rat; big endothelin-2 (1-38), human; big endothelin-2 (22-38), human; big endothelin-3, rat; biotinyl-big endothelin-1, human; and [Tyr¹²³]-prepro-endothelin (110-130), amide, human.

ETa receptor antagonist peptides including, but not limited to, [BQ-123]; [BE18257B]; [BE-18257A]/[W-7338A]; [BQ-485]; FR139317; PD-151242; and TTA-386.

ETb receptor antagonist peptides including, but not limited to, [BQ-3020]; [RES-701-3]; and [IRL-1720].

Enkephalin peptides including, but not limited to, adrenorphin, free acid; amidorphin (proenkephalin A (104-129)-NH2), bovine; BAM-12P (bovine adrenal medulla dodecapeptide); BAM-22P (bovine adrenal medulla docosapeptide); benzoyl-Phe-Ala-Arg; enkephalin; [D-Ala², D-Leu⁵]-enkephalin; [D-Ala², D-Met⁵]-enkephalin; [DAla²]-Leu-enkephalin, amide; [DAla²,Leu⁵,Arg⁶]-enkephalin; [Des-Tyr¹,DPen^{2,5}]-enkephalin; [Des-Tyr¹,DPen²,Pen⁵]-enkephalin; [Des-Tyr¹]-Leu-enkephalin; [D-Pen^{2,5}]-enkephalin; [DPen², Pen⁵]-enkephalin; enkephalinase substrate; [D-Pen², pCI-Phe⁴, D-Pen⁵]-enkephalin; Leu-enkephalin; Leu-enkephalin, amide; biotinyl-Leu-enkephalin; [D-Ala²]-Leu-enkephalin; [D-Ser²]-Leu-enkephalin-Thr (delta-receptor peptide) (DSLET); [D-Thr²]-Leu-enkephalin-Thr (DTLET); [Lys⁶]-Leu-enkephalin; [Met⁵,Arg⁶]-enkephalin; [Met⁵,Arg⁶]-enkephalin-Arg; [Met⁵,Arg⁶,Phe⁷]-enkephalin, amide; Met-enkephalin; biotinyl-Met-enkephalin; [D-Ala²]-Met-enkephalin; [D-Ala²]-Met-enkephalin, amide; Met-enkephalin-Arg-Phe; Met-enkephalin, amide; [Ala²]-Met-enkephalin, amide; [DMet²,Pro⁵]-enkephalin, amide; [DTrp²]-Met-enkephalin, amide, metorphinamide (adrenorphin); peptide B, bovine; 3200-Dalton adrenal peptide E, bovine; peptide F, bovine; preproenkephalin B 186-204, human; spinorphin, bovine; and thiorphan (D, L, 3-mercapto-2-benzylpropanoyl-glycine).

Fibronectin peptides including, but not limited to platelet factor-4 (58-70), human; echistatin (Echis carinatus); E, P, L selectin conserved region; fibronectin analog; fibronectin-binding protein; fibrinopeptide A, human; [Tyr⁰]-fibrinopeptide A, human; fibrinopeptide B, human; [Glu¹]-fibrinopeptide B, human; [Tyr¹⁵]-fibrinopeptide B, human; fibrinogen beta-chain fragment of 24-42; fibrinogen binding inhibitor peptide; fibronectin related peptide (collagen binding fragment); fibrinolysis inhibiting factor; FN-C/H-1 (fibronectin heparin-binding fragment); FN-C/H-V (fibronectin heparin-binding fragment); heparin-binding peptide; laminin penta peptide, amide; Leu-Asp-Val-NH2 (LDV-NH2), human, bovine, rat, chicken; necrofibrin, human; necrofibrin, rat; and platelet membrane glycoprotein IIB peptide 296-306.

Galanin peptides including, but not limited to, galanin, human; galanin 1-19, human; preprogalanin 1-30, human; preprogalanin 65-88, human; preprogalanin 89-123, human; galanin, porcine; galanin 1-16, porcine, rat; galanin, rat; biotinyl-galanin, rat; preprogalanin 28-67, rat; galanin 1-13-bradykinin 2-9, amide; M40, galanin 1-13-Pro-Pro-(Ala-Leu) 2-Ala-amide; C7, galanin 1-13-spantide-amide; GMAP 1-41, amide; GMAP 16-41, amide; GMAP 25-41, amide; galantide; and entero-kassinin.

Gastrin peptides including, but not limited to, gastrin, chicken; gastric inhibitory peptide (GIP), human; gastrin I, human; biotinyl-gastrin I, human; big gastrin-1, human; gastrin releasing peptide, human; gastrin releasing peptide 1-16, human; gastric inhibitory polypeptide (GIP), porcine; gastrin releasing peptide, porcine; biotinyl-gastrin releasing peptide, porcine; gastrin releasing peptide 14-27, porcine, human; little gastrin, rat; pentagastrin; gastric inhibitory peptide 1-30, porcine; gastric inhibitory peptide 1-30, amide, porcine; [Tyr⁰]-gastric inhibitory peptide 23-42, human; and gastric inhibitory peptide, rat.

Glucagon peptides including, but not limited to, [Des-His¹,Glu⁹]-glucagon, extendin-4, glucagon, human; biotinyl-glucagon, human; glucagon 19-29, human; glucagon 22-29, human; Des-His¹-[Glu⁹]-glucagon, amide; glucagon-like peptide 1, amide (preproglucagon 72-107, amide); glucagon-like peptide 1 (preproglucagon 72-108), human; glucagon-like peptide 1 (7-36) (preproglucagon 78-107, amide); glucagon-like peptide II, rat; biotinyl-glucagon-like peptide-1 (7-36) (biotinyl-preproglucagon 78-107, amide); glucagon-like peptide 2 (preproglucagon 126-159), human; oxyntomodulin/glucagon 37; and valosin (peptide VQY), porcine.

Gn-RH associated peptides (GAP) including, but not limited to, Gn-RH associated peptide 25-53, human; Gn-RH associated peptide 1-24, human; Gn-RH associated peptide 1-13, human; Gn-RH associated peptide 1-13, rat; gonadotropin releasing peptide, follicular, human; [Tyr⁰]-GAP ([Tyr⁰]-Gn-RH Precursor Peptide 14-69), human; and proopiomelanocortin (POMC) precursor 27-52, porcine.

Growth factor peptides including, but not limited to, cell growth factors; epidermal growth factors; tumor growth factor; alpha-TGF; beta-TF; alpha-TGF 34-43, rat; EGF, human; acidic fibroblast growth factor; basic fibroblast growth factor; basic fibroblast growth factor 13-18; basic fibroblast growth factor 120-125; brain derived acidic fibroblast growth factor 1-11; brain derived basic fibroblast growth factor 1-24; brain derived acidic fibroblast growth factor 102-111; [Cys(Acm^{20,31})]-epidermal growth factor 20-31; epidermal growth factor receptor peptide 985-996; insulin-like growth factor (IGF)-I, chicken; IGF-I, rat; IGF-I, human; Des (1-3) IGF-I, human; R3 IGF-I, human; R3 IGF-I, human; long R3 IGF-I, human; adjuvant peptide analog; anorexigenic peptide; Des (1-6) IGF-II, human; R6 IGF-II, human; IGF-I analogue; IGF I (24-41); IGF I (57-70); IGF I (30-41); IGF II; IGF II (33-40); [Tyr⁰]-IGF II (33-40); liver cell growth factor; midkine; midkine 60-121, human; N-acetyl, alpha-TGF 34-43, methyl ester, rat; nerve growth factor (NGF), mouse; platelet-derived growth factor; platelet-derived growth factor antagonist; transforming growth factor-alpha, human; and transforming growth factor-I, rat.

Growth hormone peptides including, but not limited to, growth hormone (hGH), human; growth hormone 1-43, human; growth hormone 6-13, human; growth hormone releasing factor, human; growth hormone releasing factor, bovine; growth hormone releasing factor, porcine; growth hormone releasing factor 1-29, amide, rat; growth hormone pro-releasing factor, human; biotinyl-growth hormone releasing factor, human; growth hormone releasing factor 1-29, amide, human; [D-Ala²]-growth hormone releasing factor 1-29, amide, human; [N-Ac-Tyr¹, D-Arg²]-GRF 1-29, amide; [His¹, Nle²⁷]-growth hormone releasing factor 1-32, amide; growth hormone releasing factor 1-37, human; growth hormone releasing factor 1-40, human; growth hormone releasing factor 1-40, amide, human; growth hormone releasing factor 30-44, amide, human; growth hormone releasing factor, mouse; growth hormone releasing factor, ovine; growth hormone releasing factor, rat; biotinyl-growth hormone releasing factor, rat; GHRP-6 ([His¹, Lys⁶]-GHRP); hexarelin (growth hormone releasing hexapeptide); and [D-Lys³]-GHRP-6.

GTP-binding protein fragment peptides including, but not limited to, [Arg⁸]-GTP-binding protein fragment, Gs alpha; GTP-binding protein fragment, G beta; GTP-binding protein fragment, GAlpha; GTP-binding protein fragment, Go Alpha; GTP-binding protein fragment, Gs Alpha; and GTP-binding protein fragment, G Alpha i2.

Guanylin peptides including, but not limited to, guanylin, human; guanylin, rat; and uroguanylin.

Inhibin peptides including, but not limited to, inhibin, bovine; inhibin, alpha-subunit 1-32, human; [Tyr⁰]-inhibin, alpha-subunit 1-32, human; seminal plasma inhibin-like peptide, human; [Tyr⁰]-seminal plasma inhibin-like peptide, human; inhibin, alpha-subunit 1-32, porcine; and [Tyr⁰]-inhibin, alpha-subunit 1-32, porcine.

Interferon peptides including, but not limited to, alpha interferon species (e.g., alpha1, alpha2, alpha2a, alpha2b, alpha2c, alpha2d, alpha3, alpha4, alpha4a, alpha4b, alpha5, alpha6, alpha74, alpha76, alphaA, alphaB, alphaC, alphaC1, alphaD, alphaE, alphaF, alphaG, alphaG, alphaH, alphaI, alphaJ1, alphaJ2, alphaK, alphaL); interferon beta species (e.g., beta1a); interferon gamma species (e.g., gamma1a, gamma1b); interferon epsilon; interferon tau; interferon omega or any analogues of interferon omega. Various analogs of gamma interferon are described in Pechenov et al. “Methods for preparation of recombinant cytokine proteins V. mutant analogues of human interferon-gamma with higher stability and activity” Protein Expr. Purif. 24:173-180 (2002), which is incorporated herein by reference in its entirety for teachings directed to preparation and testing of interferon analogues.

Insulin peptides including, but not limited to, insulin, human; insulin, porcine; IGF-I, human; insulin-like growth factor II (69-84); pro-insulin-like growth factor II (68-102), human; pro-insulin-like growth factor II (105-128), human; [Asp^{B28}]-insulin, human; [Lys^{B28}]-insulin, human; [Leu^{B28}]-insulin, human; [Val^{B28}]-insulin, human; [Ala^{B28}]-insulin, human; [Asp^{B28}, Pro^{B29}]-insulin, human; [Lys^{B28}, Pro^{B29}]-insulin, human; [Leu^{B28}, Pro^{B29}]-insulin, human; [Val^{B28}, Pro^{B29}]-insulin, human; [Ala^{B28}, Pro^{B29}]-insulin, human; [Gly^{A21}]-insulin, human; [Gly^{A21} Gln^{B3}]-insulin, human; [Ala^{A21}]-insulin, human; [Ala^{A21} Gln^{B3}]-insulin, human; [Gln^{B3}]-insulin, human; [Gln^{B30}]-insulin, human; [Gly^{A21} Glu^{B30}]-insulin, human; [Gly^{A21} Gln^{B3} Glu^{B30}]-insulin, human; [Gln^{B3} Glu^{B30}]-insulin, human; B22-B30 insulin, human; B23-B30 insulin, human; B25-B30 insulin, human; B26-B30 insulin, human; B27-B30 insulin, human; B29-B30 insulin, human; the A chain of human insulin, and the B chain of human insulin.

Interleukin peptides including, but not limited to, interleukin-1 beta 165-181, rat; and interleukin-8 (IL-8, CINC/gro), rat.

Laminin peptides including, but not limited to, laminin; alpha1 (I)-CB3 435-438, rat; and laminin binding inhibitor.

Leptin peptides including, but not limited to, leptin 93-105, human; leptin 22-56, rat; Tyr-leptin 26-39, human; and leptin 116-130, amide, mouse.

Leucokinin peptides including, but not limited to, leucomyosuppressin (LMS); leucopyrokinin (LPK); leucokinin I; leucokinin II; leucokinin III; leucokinin IV; leucokinin VI; leucokinin VII; and leucokinin VIII.

Luteinizing hormone-releasing hormone peptides including, but not limited to, antide; Gn-RH II, chicken; luteinizing hormone-releasing hormone (LH-RH) (GnRH); biotinyl-LH-RH; cetrorelix (D-20761); [D-Ala⁶]-LH-RH; [Gln⁸]-LH-RH (Chicken LH-RH); [DLeu⁶, Val⁷] LH-RH 1-9, ethyl amide; [D-Lys⁶]-LH-RH; [D-Phe², Pro³, D-Phe⁶]-LH-RH; [DPhe², DAla⁶] LH-RH; [Des-Gly¹⁰]-LH-RH, ethyl amide; [D-Ala⁶, Des-Gly¹⁰]-LH-RH, ethyl amide; [DTrp⁶]-LH-RH, ethyl amide; [D-Trp⁶, Des-Gly¹⁰]-LH-RH, ethyl amide (Deslorelin); [DSer(But)⁶, Des-Gly¹⁰]-LH-RH, ethyl amide; ethyl amide; leuprolide; LH-RH 4-10; LH-RH 7-10; LH-RH, free acid; LH-RH, lamprey; LH-RH, salmon; [Lys⁸]-LH-RH; [Trp⁷,Leu⁸] LH-RH, free acid; and [(t-Bu)DSer⁶, (Aza)Gly¹⁰]-LH-RH.

Mastoparan peptides including, but not limited to, mastoparan; mas7; mas8; mas17; and mastoparan X.

Mast cell degranulating peptides including, but not limited to, mast cell degranulating peptide HR-1; and mast cell degranulating peptide HR-2.

Melanocyte stimulating hormone (MSH) peptides including, but not limited to, [Ac-Cys⁴,DPhe⁷,Cys¹⁰] alpha-MSH 4-13, amide; alpha-melanocyte stimulating hormone; alpha-MSH, free acid; beta-MSH, porcine; biotinyl-alpha-melanocyte stimulating hormone; biotinyl-[Nle⁴, D-Phe⁷] alpha-melanocyte stimulating hormone; [Des-Acetyl]-alpha-MSH; [DPhe⁷]-alpha-MSH, amide; gamma-1-MSH, amide; [Lys⁰]-gamma-1-MSH, amide; MSH release inhibiting factor, amide; [Nle⁴]-alpha-MSH, amide; [Nle⁴, D-Phe⁷]-alpha-MSH; N-Acetyl, [Nle⁴,DPhe⁷] alpha-MSH 4-10, amide; beta-MSH, human; and gamma-MSH.

Morphiceptin peptides including, but not limited to, morphiceptin (beta-casomorphin 1-4 amide); [D-Pro⁴]-morphiceptin; and [N-MePhe³,D-Pro⁴]-morphiceptin.

Motilin peptides including, but not limited to, motilin, canine; motilin, porcine; biotinyl-motilin, porcine; and [Leu¹³]-motilin, porcine.

Neuro-peptides including, but not limited to, Ac-Asp-Glu; achatina cardio-excitatory peptide-1 (ACEP-1) (Achatina fulica); adipokinetic hormone (AKH) (Locust); adipokinetic hormone (Heliothis zea and Manduca sexta); alytesin; Tabanus atratus adipokinetic hormone (Taa-AKH); adipokinetic hormone II (Locusta migratoria); adipokinetic hormone II (Schistocera gregaria); adipokinetic hormone III (AKH-3); adipokinetic hormone G (AKH-G) (Gryllus bimaculatus); allatotropin (AT) (Manduca sexta); allatotropin 6-13 (Manduca sexta); APGW amide (Lymnaea stagnalis); buccalin; cerebellin; [Des-Ser¹]-cerebellin; corazonin (American Cockroach Periplaneta americana); crustacean cardioactive peptide (CCAP); crustacean erythrophore; DF2 (Procambarus clarkii); diazepam-binding inhibitor fragment, human; diazepam binding inhibitor fragment (ODN); eledoisin related peptide; FMRF amide (molluscan cardioexcitatory neuro-peptide); Gly-Pro-Glu (GPE), human; granuliberin R; head activator neuropeptide; [His⁷]-corazonin; stick insect hypertrehalosaemic factor II; Tabanus atratus hypotrehalosemic hormone (Taa-HoTH); isoguvacine hydrochloride; bicuculline methiodide; piperidine-4-sulphonic acid; joining peptide of proopiomelanocortin (POMC), bovine; joining peptide, rat; KSAYMRF amide (P. redivivus); kassinin; kinetensin; levitide; litorin; LUQ 81-91 (Aplysia californica); LUQ 83-91 (Aplysia californica); myoactive peptide I (Periplanetin CC-1) (Neuro-hormone D); myoactive peptide II (Periplanetin CC-2); myomodulin; neuron specific peptide; neuron specific enolase 404-443, rat; neuropeptide FF; neuropeptide K, porcine; NEI (prepro-MCH 131-143) neuropeptide, rat; NGE (prepro-MCH 110-128) neuropeptide, rat; NFI (Procambarus clarkii); PBAN-1 (Bombyx mori); Hez-PBAN (Heliothis zea); SCPB (cardioactive peptide from aplysia); secretoneurin, rat; uperolein; urechistachykinin I; urechistachykinin II; xenopsin-related peptide I; xenopsin-related peptide II; pedal peptide (Pep), aplysia; peptide F1, lobster; phyllomedusin; polistes mastoparan; proctolin; ranatensin; Ro I (Lubber Grasshopper, Romalea microptera); Ro II (Lubber Grasshopper, Romalea microptera); SALMF amide 1 (S1); SALMF amide 2 (S2); and SCPA.

Neuropeptide Y (NPY) peptides including, but not limited to, [Leu³¹,Pro³⁴]-neuropeptide Y, human; neuropeptide F (Moniezia expansa); B1BP3226 NPY antagonist; Bis (31/31′) {[Cys³¹, TrP³², Nva³⁴] NPY 31-36}; neuropeptide Y, human, rat; neuropeptide Y1-24 amide, human; biotinyl-neuropeptide Y; [D-Tyr^{27,36}, D-Thr³²]-NPY 27-36; Des 10-17 (cyclo 7-21) [Cys^{7,21}, Pro³⁴]-NPY; C2-NPY; [Leu³¹, Pro³⁴] neuropeptide Y, human; neuropeptide Y, free acid, human; neuropeptide Y, free acid, porcine; prepro NPY 68-97, human; N-acetyl-[Leu²⁸, Leu³¹] NPY 24-36; neuropeptide Y, porcine; [D-TrP³²]-neuropeptide Y, porcine; [D-TrP³²] NPY 1-36, human; [Leu¹⁷,DTrP³²] neuropeptide Y, human; [Leu³¹, Pro³⁴]-NPY, porcine; NPY 2-36, porcine; NPY 3-36, human; NPY 3-36, porcine; NPY 13-36, human; NPY 13-36, porcine; NPY 16-36. porcine; NPY 18-36, porcine; NPY 20-36; NFY 22-36; NPY 26-36; [Pro³⁴]-NPY 1-36, human; [Pro³⁴]-neuropeptide Y, porcine; PYX-1; PYX-2; T4-[NPY(33-36)]4; and Tyr(OMe)²¹]-neuropeptide Y, human.

Neurotropic factor peptides including, but not limited to, glial derived neurotropic factor (GDNF); brain derived neurotropic factor (BDNF); and ciliary neurotropic factor (CNTF).

Orexin peptides including, but not limited to, orexin A; orexin B, human; orexin B, rat, mouse.

Opioid peptides including, but not limited to, alpha-casein fragment 90-95; BAM-18P; casomokinin L; casoxin D; crystalline; DALDA; dermenkephalin (deltorphin) (Phylomedusa sauvagei); [D-Ala²]-deltorphin I; [D-Ala²]-deltorphin II; endomorphin-1; endomorphin-2; kyotorphin; [DArg²]-kyotorphin; morphin tolerance peptide; morphine modulating peptide, C-terminal fragment; morphine modulating neuropeptide (A-18-F-NH2); nociceptin [orphanin FQ] (ORL1 agonist); TIPP; Tyr-MIF-1; Tyr-W-MIF-1; valorphin; LW-hemorphin-6, human; Leu-valorphin-Arg; and Z-Pro-D-Leu.

Oxytocin peptides including, but not limited to, [Asu⁶]-oxytocin; oxytocin; biotinyl-oxytocin; [Thr⁴, Gly⁷]-oxytocin; and tocinoic acid ([Ile³]-pressinoic acid).

PACAP (pituitary adenylating cyclase activating peptide) peptides including, but not limited to, PACAP 1-27, human, ovine, rat; PACAP (1-27)-Gly-Lys-Arg-NH2, human; [Des-Gln¹⁶]-PACAP 6-27, human, ovine, rat; PACAP38, frog; PACAP27-NH2, human, ovine, rat; biofinyl-PACAP27-NH2, human, ovine, rat; PACAP 6-27, human, ovine, rat; PACAP38, human, ovine, rat; biotinyl-PACAP38, human, ovine, rat; PACAP 6-38, human, ovine, rat; PACAP27-NH2, human, ovine, rat; biotinyl-PACAP27-NH2, human, ovine, rat; PACAP 6-27, human, ovine, rat; PACAP38, human, ovine, rat; biotinyl-PACAP38, human, ovine, rat; PACAP 6-38, human, ovine, rat; PACAP38 16-38, human, ovine, rat; PACAP38 31-38, human, ovine, rat; PACAP38 31-38, human, ovine, rat; PACAP-related peptide (PRP), human; and PACAP-related peptide (PRP), rat.

Pancreastatin peptides including, but not limited to, chromostatin, bovine; pancreastatin (hPST-52) (chromogranin A 250-301, amide); pancreastatin 24-52 (hPST-29), human; chromogranin A 286-301, amide, human; pancreastatin, porcine; biotinyl-pancreastatin, porcine; [Nle⁸]-pancreastatin, porcine; [Tyr⁰,Nle⁸]-pancreastatin, porcine; [Tyr⁰]-pancreastatin, porcine; parastatin 1-19 (chromogranin A 347-365), porcine; pancreastatin (chromogranin A 264-314-amide, rat; biotinyl-pancreastatin (biotinyl-chromogranin A 264-314-amide; [Tyr⁰]-pancreastatin, rat; pancreastatin 26-51, rat; and pancreastatin 33-49, porcine.

Pancreatic polypeptides including, but not limited to, pancreatic polypeptide, avian; pancreatic polypeptide, human; C-fragment pancreatic polypeptide acid, human; C-fragment pancreatic polypeptide amide, human; pancreatic polypeptide (Rana temporaria); pancreatic polypeptide, rat; and pancreatic polypeptide, salmon.

Parathyroid hormone peptides including, but not limited to, [Asp⁷⁶]-parathyroid hormone 39-84, human; [Asp⁷⁶]-parathyroid hormone 53-84, human; [Asn⁷⁶]-parathyroid hormone 1-84, hormone; [Asn⁷⁶]-parathyroid hormone 64-84, human; [Asn⁸, Leu¹⁸]-parathyroid hormone 1-34, human; [Cys^{5,28}]-parathyroid hormone 1-34, human; hypercalcemia malignancy factor 1-40; [Leu¹⁸]-parathyroid hormone 1-34, human; [Lys(biotinyl)¹³, Nle^{8,18}, Tyr³⁴]-parathyroid hormone 1-34 amide; [Nle^{8,18}, Tyr³⁴]-parathyroid hormone 1-34 amide; [Nle^{8,18}, Tyr³⁴]-parathyroid hormone 3-34 amide, bovine; [Nle^{8,18}, Tyr³⁴]-parathyroid hormone 1-34, human; [Nle^{8,18}, Tyr³⁴]-parathyroid hormone 1-34 amide, human; [Nle^{8,18}, Tyr³⁴]-parathyroid hormone 3-34 amide, human; [Nle^{8,18}, Tyr³⁴]-parathyroid hormone 7-34 amide, bovine; [Nle^{8,21}, Tyr³⁴]-parathyroid hormone 1-34 amide, rat; parathyroid hormone 44-68, human; parathyroid hormone 1-34, bovine; parathyroid hormone 3-34, bovine; parathyroid hormone 1-31 amide, human; parathyroid hormone 1-34, human; parathyroid hormone 13-34, human; parathyroid hormone 1-34, rat; parathyroid hormone 1-38, human; parathyroid hormone 1-44, human; parathyroid hormone 28-48, human; parathyroid hormone 39-68, human; parathyroid hormone 39-84, human; parathyroid hormone 53-84, human; parathyroid hormone 69-84, human; parathyroid hormone 70-84, human; [Pro³⁴]-peptide YY (PYY), human; [Tyr⁰]-hypercalcemia malignancy factor 1-40; [Tyr⁰]-parathyroid hormone 1-44, human; [Tyr⁰]-parathyroid hormone 1-34, human; [Tyr¹]-parathyroid hormone 1-34, human; [Tyr²⁷]-parathyroid hormone 27-48, human; [Tyr³⁴]-parathyroid hormone 7-34 amide, bovine; [Tyr⁴³]-parathyroid hormone 43-68, human; [Tyr⁵², Asn⁷⁶]-parathyroid hormone 52-84, human; and [Tyr⁶³]-parathyroid hormone 63-84, human.

Parathyroid hormone (PTH)-related peptides including, but not limited to, PTHrP ([Tyr³⁶]-PTHrP 1-36 amide), chicken; hHCF-(1-34)-NH2 (humoral hypercalcemic factor), human; PTH-related protein 1-34, human; biotinyl-PTH-related protein 1-34, human; [Tyr⁰]-PTH-related protein 1-34, human; [Tyr³⁴]-PTH-related protein 1-34 amide, human; PTH-related protein 1-37, human; PTH-related protein 7-34 amide, human; PTH-related protein 38-64 amide, human; PTH-related protein 67-86 amide, human; PTH-related protein 107-111, human, rat, mouse; PTH-related protein 107-111 free acid; PTH-related protein 107-138, human; and PTH-related protein 109-111, human.

Peptide T peptides including, but not limited to, peptide T; [D-Ala¹]-peptide T; and [D-Ala¹]-peptide T amide.

Prolactin-releasing peptides including, but not limited to, prolactin-releasing peptide 31, human; prolactin-releasing peptide 20, human; prolactin-releasing peptide 31, rat; prolactin-releasing peptide 20, rat; prolactin-releasing peptide 31, bovine; and prolactin-releasing peptide 20, bovine.

Peptide YY (PYY) peptides including, but not limited to, PYY, human; PYY 3-36, human; biotinyl-PYY, human; PYY, porcine, rat; and [Leu³¹, Pro³⁴]-PYY, human.

Renin substrate peptides including, but not limited to, acetyl, angiotensinogen 1-14, human; angiotensinogen 1-14, porcine; renin substrate tetradecapeptide, rat; [Cys⁸]-renin substrate tetradecapeptide, rat; [Leu⁸]-renin substrate tetradecapeptide, rat; and [Val⁸]-renin substrate tetradecapeptide, rat.

Secretin peptides including, but not limited to, secretin, canine; secretin, chicken; secretin, human; biotinyl-secretin, human; secretin, porcine; and secretin, rat.

Somatostatin (GIF) peptides including, but not limited to, BIM-23027; biotinyl-somatostatin; biotinylated cortistatin 17, human; cortistatin 14, rat; cortistatin 17, human; [Tyr⁰]-cortistatin 17, human; cortistatin 29, rat; [D-Trp⁸]-somatostatin; [DTrp⁸,DCys¹⁴]-somatostatin; [DTrp⁸,Tyr¹¹]-somatostatin; [D-Trp¹¹]-somatostatin; NTB (Naltriben); [Nle⁸]-somatostatin 1-28; octreotide (SMS 201-995); prosomatostatin 1-32, porcine; [Tyr⁰]-somatostatin; [Tyr¹]-somatostatin; [Tyr¹]-somatostatin 28 (1-14); [Tyr¹¹]-somatostatin; [Tyr⁰, D-Trp⁸]-somatostatin; somatostatin; somatostatin antagonist; somatostatin-25; somatostatin-28; somatostatin 28 (1-12); biotinyl-somatostatin-28; [Tyr⁰]-somatostatin-28; [Leu⁸, D-Trp²², Tyr²⁵]-somatostatin-28; biotinyl-[Leu⁸, D-Trp²², Tyr²⁵]-somatostatin-28; somatostatin-28 (1-14); and somatostatin analog, RC-160.

Substance P peptides including, but not limited to, G protein antagonist-2; Ac-[Arg⁶, Sar⁹, Met(02)¹¹]-substance P 6-11; [Arg³]-substance P; Ac-Trp-3,5-bis(trifluoromethyl) benzyl ester; Ac-[Arg⁶, Sar⁹, Met(O2)¹¹]-substance P 6-11; [D-Ala⁴]-substance P 4-11; [Tyr⁶, D-Phe⁷, D-His⁹]-substance P 6-11 (sendide); biotinyl-substance P; biotinyl-NTE[Arg³]-substance P; [Tyr⁸]-substance P; [Sar⁹, Met(O2)¹¹]-substance P; [D-Pro², D-Trp^{7,9}]-substance P; [D-Pro⁴, 0-Trp^{7,9}]-substance P 4-11; substance P 4-11; [DTrp^{2,7,9}]-substance P; [(Dehydro)Pro^{2,4}, Pro⁹]-substance P; [Dehydro-Pro⁴]-substance P 4-11; [Glp⁵,(Me)Phe⁸,Sar⁹]-substance P 5-11; [Glp⁵,Sar⁹]-substance P 5-11; [Glp⁵]-substance P 5-11; hepta-substance P (substance P 5-11); hexa-substance P(substance P 6-11); [MePhe⁸,Sar⁹]-substance P; [Nle¹¹]-substance P; Octa-substance P(substance P 4-11); [pGlu¹]-hexa-substance P ([pGlu⁶]-substance P 6-11); [pGlu⁶, D-Pro⁹]-substance P 6-11; [(pNO2)Phe⁷Nle¹¹]-substance P; penta-substance P (substance P 7-11); [Pro⁹]-substance P; GR73632, substance P 7-11; [Sar⁴]-substance P 4-11; [Sar⁹]-substance P; septide ([pGlu⁶, Pro⁹]-substance P 6-11); spantide I; spantide II; substance P; substance P, cod; substance P, trout; substance P antagonist; substance P-Gly-Lys-Arg; substance P1-4; substance P1-6; substance P1-7; substance P1-9; deca-substance P (substance P 2-11); nona-substance P (substance P 3-11); substance P tetrapeptide (substance P 8-11); substance P tripeptide (substance P 9-11); substance P, free acid; substance P methyl ester; and [Tyr⁸,Nle¹¹] substance P.

Tachykinin peptides including, but not limited to, [Ala⁵, beta-Ala⁸] neurokinin A 4-10; eledoisin; locustatachykinin I (Lom-TK-I) (Locusta migratoria); locustatachykinin II (Lom-TK-II) (Locusta migratoria); neurokinin A 4-10; neurokinin A (neuromedin L, substance K); neurokinin A, cod and trout; biotinyl-neurokinin A (biotinyl-neuromedin L, biotinyl-substance K); [Tyr⁰]-neurokinin A; [Tyr⁶]-substance K; FR64349; [Lys³, Gly⁸-(R)-gamma-lactam-Leu⁹]-neurokinin A 3-10; GR83074; GR87389; GR94800; [Beta-Ala⁸]-neurokinin A 4-10; [Nle¹⁰]-neurokinin A 4-10; [Trp⁷, beta-Ala⁸]-neurokinin A 4-10; neurokinin B (neuromedin K); biotinyl-neurokinin B (biotinyl-neuromedin K); [MePhe⁷]-neurokinin B; [Pro⁷]-neurokinin B; [Tyr⁰]-neurokinin B; neuromedin B, porcine; biotinyl-neuromedin B, porcine; neuromedin B-30, porcine; neuromedin B-32, porcine; neuromedin B receptor antagonist; neuromedin C, porcine; neuromedin N, porcine; neuromedin (U-8), porcine; neuromedin (U-25), porcine; neuromedin U, rat; neuropeptide-gamma (gamma-preprotachykinin 72-92); PG-KII; phyllolitorin; [Leu⁸]-phyllolitorin (Phyllomedusa sauvagei); physalaemin; physalaemin 1-11; scyliorhinin II, amide, dogfish; senktide, selective neurokinin B receptor peptide; [Ser²]-neuromedin C; beta-preprotachykinin 69-91, human; beta-preprotachykinin 111-129, human; tachyplesin I; xenopsin; and xenopsin 25 (xenin 25), human.

Thyrotropin-releasing hormone (TRH) peptides including, but not limited to, biotinyl-thyrotropin-releasing hormone; [Glu¹]-TRH; His-Pro-diketopiperazine; [3-Me-His²]-TRH; pGlu-Gln-Pro-amide; pGlu-His; [Phe²]-TRH; prepro TRH 53-74; prepro TRH 83-106; prepro-TRH 160-169 (Ps4, TRH-potentiating peptide); prepro-TRH 178-199; thyrotropin-releasing hormone (TRH); TRH, free acid; TRH-SH Pro; and TRH precursor peptide.

Toxin peptides including, but not limited to, omega-agatoxin TK; agelenin, (spider, Agelena opulenta); apamin (honeybee, Apis mellifera); calcicudine (CaC) (green mamba, Dedroaspis angusficeps); calciseptine (black mamba, Dendroaspis polylepis polylepis); charybdotoxin (ChTX) (scorpion, Leiurus quinquestriatus var. hebraeus); chlorotoxin; conotoxin GI (marine snail, Conus geographus); conotoxin GS (marine snail, Conus geographus); conotoxin MI (Marine Conus magus); alpha-conotoxin EI, Conus ermineus; alpha-conotoxin SIA; alpha-conotoxin Iml; alpha-conotoxin SI (cone snail, Conus striatus); micro-conotoxin GIIIB (marine snail, Conus geographus); omega-conotoxin GVIA (marine snail, Conus geographus); omega-conotoxin MVIIA (Conus magus); omega-conotoxin MVIIC (Conus magus); omega-conotoxin SVIB (cone snail, Conus striatus); endotoxin inhibitor; geographutoxin I (GTX-I) (μ-Conotoxin GIIIA); iberiotoxin (IbTX) (scorpion, Buthus tamulus); kaliotoxin 1-37; kaliotoxin (scorpion, Androct-onus mauretanicus mauretanicus); mast cell-degranulating peptide (MCD-peptide, peptide 401); margatoxin (MgTX) (scorpion, Centruriodes Margaritatus); neurotoxin NSTX-3 (pupua new guinean spider, Nephilia maculata); PLTX-II (spider, Plectreurys tristes); scyllatoxin (leiurotoxin I); and stichodactyla toxin (ShK).

Vasoactive intestinal peptides (VIP/PHI) including, but not limited to, VIP, human, porcine, rat, ovine; VIP-Gly-Lys-Arg-NH2; biotinyl-PHI (biotinyl-PHI-27), porcine; [Glp¹⁶] VIP 16-28, porcine; PHI (PHI-27), porcine; PHI (PHI-27), rat; PHM-27 (PHI), human; prepro VIP 81-122, human; preproVIP/PHM 111-122; prepro VIP/PHM 156-170; biotinyl-PHM-27 (biotinyl-PHI), human; vasoactive intestinal contractor (endothelin-beta); vasoactive intestinal octacosa-peptide, chicken; vasoactive intestinal peptide, guinea pig; biotinyl-VIP, human, porcine, rat; vasoactive intestinal peptide 1-12, human, porcine, rat; vasoactive intestinal peptide 10-28, human, porcine, rat; vasoactive intestinal peptide 11-28, human, porcine, rat, ovine; vasoactive intestinal peptide (cod, Gadus morhua); vasoactive intestinal peptide 6-28; vasoactive intestinal peptide antagonist; vasoactive intestinal peptide antagonist ([Ac-Tyr¹, D-Phe²]-GHRF 1-29 amide); vasoactive intestinal peptide receptor antagonist (4-Cl-D-Phe⁶, Leu¹⁷]-VIP); and vasoactive intestinal peptide receptor binding inhibitor, L-8-K.

Vasopressin (ADH) peptides including, but not limited to, vasopressin; [Asu^{1,6},Arg⁸]-vasopressin; vasotocin; [Asu^{1,6},Arg⁸]-vasotocin; [Lys⁸]-vasopressin; pressinoic acid; [Arg⁸]-desamino vasopressin desglycinamide; [Arg⁸]-vasopressin (AVP); [Arg⁸]-vasopressin desglycinamide; biotinyl-[Arg⁸]-vasopressin (biotinyl-AVP); [D-Arg⁸]-vasopressin; desamino-[Arg⁸]-vasopressin; desamino-[D-Arg⁸]-vasopressin (DDAVP); [deamino-[D-3-(3′-pyridyl-Ala)]-[Arg⁸]-vasopressin; [1-(beta-Mercapto-beta, beta-cyclopentamethylene propionic acid), 2-(O-methyl)tyrosine]-[Arg⁸]-vasopressin; vasopressin metabolite neuropeptide [pGlu⁴, Cys⁶]; vasopressin metabolite neuropeptide [pGlu⁴, Cys⁶]; [Lys⁸]-deamino vasopressin desglycinamide; [Lys⁸]-vasopressin; [Mpr¹,Val⁴,DArg⁸]-vasopressin; [Phe², Ile³, Orn⁸]-vasopressin ([Phe², Orn⁸]-vasotocin); [Arg⁸]-vasotocin; and [d(CH2)5, Tyr(Me)², Orn⁸]-vasotocin.

Virus related peptides including, but not limited to, fluorogenic human CMV protease substrate; HCV core protein 59-68; HCV NS4A protein 18-40 (JT strain); HCV NS4A protein 21-34 (JT strain); hepatitis B virus receptor binding fragment; hepatitus B virus pre-S region 120-145; [Ala¹²⁷]-hepatitus B virus pre-S region 120-131; herpes virus inhibitor 2; HIV envelope protein fragment 254-274; HIV gag fragment 129-135; HIV substrate; P 18 peptide; peptide T; [3,5 diiodo-Tyr⁷] peptide T; R15K HIV-1 inhibitory peptide; T20; T21; V3 decapeptide P 18-110; and virus replication inhibiting peptide.

While certain analogs, fragments, and/or analog fragments of the various polypeptides have been described above, it is to be understood that other analogs, fragments, and/or analog fragments that retain all or some of the activity of the particular polypeptide may also be useful in embodiments of the present invention. Analogs may be obtained by various means, as will be understood by those skilled in the art. For example, certain amino acids may be substituted for other amino acids in a polypeptide without appreciable loss of interactive binding capacity with structures such as, for example, antigen-binding regions of antibodies or binding sites on substrate molecules. As the interactive capacity and nature of a polypeptide drug defines its biological functional activity, certain amino acid sequence substitutions can be made in the amino acid sequence and nevertheless remain a polypeptide with like properties.

g) Oligonucleotide Agents

The active agents can also be in the form of oligonucleotides, including oligoribonucleotides useful for prophylactic, palliative or therapeutic purposes, including gene therapy and the treatment of cancer, such as colon cancer.

An oligonucleotide is a polymer of a repeating unit generically known as a nucleotide. An unmodified (naturally occurring) nucleotide has three components: (1) a nitrogen-containing heterocyclic base linked by one of its nitrogen atoms to (2) a 5-pentofuranosyl sugar and (3) a phosphate esterified to one of the 5′ or 3′ carbon atoms of the sugar. When incorporated into an oligonucleotide chain, the phosphate of a first nucleotide is also esterified to an adjacent sugar of a second, adjacent nucleotide via a 3′-5′ phosphate linkage. Nucleotides are nucleosides that further include a phosphate group covalently linked to the sugar portion of the nucleoside. In forming oligonucleotides, the phosphate groups covalently link adjacent nucleosides to one another to form a linear polymeric compound. The respective ends of this linear polymeric structure can be further joined to form a circular structure, however, within the context of the invention, open linear structures are generally preferred.

Oligonucleotides can include nucleotide sequences sufficient in identity and number to effect specific hybridization with a particular nucleic acid. Such oligonucleotides which specifically hybridize to a portion of the sense strand of a gene are commonly described as “antisense.” In the context of the invention, “hybridization” means hydrogen bonding, which may be Watson-Crick, Hoogsteen or reversed Hoogsteen hydrogen bonding, between complementary nucleotides. For example, adenine and thymine are complementary nucleobases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other.

The term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent intersugar (backbone) linkages as well as oligonucleotides having non-naturally-occurring portions which function similarly. Generally, oligonucleotides formulated in the compositions of the invention may be from about 8 to about 100 nucleotides in length, more preferably from about 10 to about so nucleotides in length, and most preferably from about 10 about 25 nucleotides in length.

Oligonucleotides that are formulated in the compositions of the invention include antisense compounds and other bioactive oligonucleotides. A discussion of antisense oligonucleotides and some desirable modifications can be found in De Mesmaeker et al. (Acc. Chem. Res., 1995, 28, 366).

As used herein, antisense compounds include antisense oligonucleotides, antisense peptide nucleic acids (PNAs), ribozymes and EGSs. In antisense modulation of messenger RNA (mRNA), hybridization of an antisense compound with its mRNA target interferes with the normal role of mRNA and causes a modulation of its function in cells. The functions of mRNA to be interfered with include all vital functions such as translocation of the RNA to the site for protein translation, actual translation of protein from the RNA, splicing of the RNA to yield one or more mRNA species, turnover or degradation of the mRNA and possibly even independent catalytic activity which may be engaged in by the RNA. The overall effect of such interference with mRNA function is modulation of the expression of a protein, wherein “modulation” means either an increase (stimulation) or a decrease (inhibition) in the expression of the protein. In the context of the present invention, inhibition is the preferred form of modulation of gene expression.

Antisense compounds can exert their effect by a variety of means. One such means is the antisense-mediated direction of an endogenous nuclease, such as RNase H in eukaryotes or RNase P in prokaryotes, to the target nucleic acid (Chiang et al., J. Biol. Chem., 1991, 266, 18162; Forster et al., Science, 1990, 249, 783).

The sequences that recruit RNase P are known as External Guide Sequences, hence the abbreviation “EGS” (Guerrier-Takada et al., Proc. Natl. Acad. Sci. USA, 1997, 94, 8468). Another means involves covalently linking a synthetic moiety having nuclease activity to an oligonucleotide having an antisense sequence, rather than relying upon recruitment of an endogenous nuclease. Synthetic moieties having nuclease activity include, but are not limited to, enzymatic RNAs, lanthanide ion complexes, and the like (Haseloff et al., Nature, 1988, 334, 585; Baker et al., J. Am. Chem. Soc., 1997, 119, 8749).

As used herein, the term “antisense compound” also includes ribozymes, synthetic RNA molecules and derivatives thereof that catalyze highly specific endoribonuclease reactions (see, generally, U.S. Pat. No. 5,543,508 to Haseloff et al. and U.S. Pat. No. 5,545,729 to Goodchild et al.).

The antisense compounds formulated in the compositions of the invention (1) can be from about 8 to about 100 nucleotides in length, more preferably from about 10 to about 30 nucleotides in length, (2) are targeted to a nucleic acid sequence required for the expression of a gene from a mammal, including a human, and (3), when contacted with cells expressing the target gene, modulate its expression. Due to the biological activity of the gene product encoded by the target gene, modulation of its expression has the desirable result of providing specific prophylactic, palliative and/or therapeutic effects.

It is understood in the art that the nucleobase sequence of an oligonucleotide or other antisense compound need not be 100% complementary to its target nucleic acid sequence to be specifically hybridizable. An antisense compound is specifically hybridizable to its target nucleic acid when there is a sufficient degree of complementarity to avoid non-specific binding of the oligonucleotide to non-target sequences under conditions in which specific binding is desired, i.e., under physiological conditions in the case of in vivo assays or therapeutic treatment, or, in the case of in vitro assays, under assay conditions.

Other bioactive oligonucleotides include aptamers and molecular decoys. As used herein, the term is meant to refer to any oligonucleotide (including a PNA) that (1) provides a prophylactic, palliative or therapeutic effect to an animal in need thereof and (2) acts by a non-antisense mechanism, i.e., by some means other than by hybridizing to a nucleic acid.

The name aptamer has been coined by Ellington et al. (Nature, 1990, 346, 818) to refer to nucleic acid molecules that fit and therefore bind with significant specificity to non-nucleic acid ligands such as peptides, proteins and small molecules such as drugs and dyes. Because of these specific ligand binding properties, nucleic acids and oligonucleotides that may be classified as aptamers may be readily purified or isolated via affinity chromatography using columns that bear immobilized ligand. Aptamers may be nucleic acids that are relatively short to those that are as large as a few hundred nucleotides. For example, RNA aptamers that are 155 nucleotides long and that bind dyes such as Cibacron Blue and Reactive Blue 4 with good selectivity have been reported (Ellington et al., Nature, 1990, 346, 818). While RNA molecules were first referred to as aptamers, the term as used in the present invention refers to any nucleic acid or oligonucleotide that exhibits specific binding to small molecule ligands including, but not limited to, DNA, RNA, DNA derivatives and conjugates, RNA derivatives and conjugates, modified oligonucleotides, chimeric oligonucleotides, and gapmers (see, e.g., U.S. Pat. No. 5,523,3B9, to Ecker et al., issued Jun. 4, 1996 and incorporated herein by reference).

Molecular decoys are short double-stranded nucleic acids (including single-stranded nucleic acids designed to “fold back” on themselves) that mimic a site on a nucleic acid to which a factor, such as a protein, binds. Such decoys are expected to competitively inhibit the factor; that is, because the factor molecules are bound to an excess of the decoy, the concentration of factor bound to the cellular site corresponding to the decoy decreases, with resulting therapeutic, palliative or prophylactic effects. Methods of identifying and constructing decoy molecules are described in, e.g., U.S. Pat. No. 5,716,780 to Edwards et al.

Another type of bioactive oligonucleotide is an RNA-DNA hybrid molecule that can direct gene conversion of an endogenous nucleic acid (Cole-Strauss et al., Science, 1996, 273, 1386).

Preferred modified oligonucleotide backbones include, for example, phosphorothioates, chiral phosphorothioates, phosphoro-dithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalklyphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Any of the preceding bioactive oligonucleotides can be formulated into the drug delivery device of the invention and used for prophylactic or therapeutic purposes. The oligonucleotides can be stabilized through complexation, for example, with cationic lipids such as Lipoplexe or cationic polymers such as Polyplexe.

h) Diagnostic Agents

Medical imaging is the non-invasive or non-surgical visualization of internal organs or processes. Representative diagnostic methods include X-rays, magnetic resonance imaging (MRI), radionuclides or nuclear medicine, and ultrasound.

Radionuclides are nuclei that decay by dissipating excess energy (parent) to become stable (daughter) by energy emission in form of particulate or electromagnetic radiation. Fluoroscopy is a fluorescent screen that detects gamma or X rays, which are imaged by a TV camera to afford real time images of organs in motion by using contrast agents, such as PCTA. CAT—Computed axial tomography—takes advantage of small differences in tissue radiographic density to create an image. The colon is often imaged using a lower GI series of a barium enema to conduct a radiographic study of the large bowel colon and rectum.

Technetium is a common radiolabel. Other radiolabeled compounds include iodine radiolabels, such as iobenguane sulfate ¹³¹I, sodium ¹²³iodine, sodium ¹³¹iodine, and indium labels, such as ¹¹¹In radiolabels, indium chloride, and indium satumomabpendetide. Imaging contrast agents include iron-containing contrast agents such as ferumoxides and dentritic gadolinium.

### II. Methods of Preparing the Polyethyleneimine-Reticulated Pectin Beads

The polyethyleneimine-reticulated pectin beads can be prepared using methods known to those of skill in the art, including by mixing the active agent in a pectin solution, crosslinking the pectin with a metal cation such as calcium to form pectin beads that encapsulate the active agent, and reticulating the beads with a solution of polyethyleneimine.

Typically, the aqueous pectin solution includes the active ingredient at a concentration of 0.5 to 5% (v/v), and this solution is ideally added dropwise to a solution of calcium chloride to form calcium pectinate beads, which are then recovered and introduced to an aqueous solution of polyethyleneimine. The pectin solution is advantageously from 4 to 10% (m/v), preferably 4 to 7%, the calcium chloride solution is advantageously from 2 to 10% (m/v), and the polyethyleneimine solution is advantageously from 0.5 to 2% (m/v). More preferably, the pectin solution is about 6% (m/v), the calcium chloride solution is about 6% (m/v), and the polyethylenimine solution is about 0.6 to 1% (m/v), preferably about 0.8% (m/v), although in any case, the amount of polyethyleneimine is advantageously selected to provide reticulated pectin beads that survive in the gastrointestinal tract until they reach the colon, and that are sufficiently degraded in the colon to provide effective release of the active agent.

The pectin beads are advantageously stirred in the calcium chloride solution under slow agitation for between 10 minutes and 1 hour, preferably for about 20 minutes. The beads are reticulated with polyethyleneimine under slow agitation for 15 to 40 minutes, preferably for 20 minutes. After recovering the pectin beads, they are dried at a temperature of between 20 and 40° C. for 30 min to 10 hours, preferably at 37° C. for 2 hours. The diameter of the particles is between about 800 and 1500 μm, preferably between about 1000 and 1200 μm.

When the active agent is a beta-lactamase, the encapsulation yields are between 50 and 90% or 3-6 UI/beads of beta-lactamases, activity expressed in substrate benzylpenicillin, whether the pectin is amidated or not.

### III. Formation of Drug Delivery Devices Including the Pectin Beads

The pectin beads can be collected, and combined with appropriate excipients and formulated into a variety of oral drug delivery devices. For example, the beads can be combined with a solid excipient, and tableted, or included in a capsule.

The pectin beads can also be combined with liquid/gel excipients which do not degrade the pectin beads, and the mixture/dispersion can be incorporated into a capsule, such as a gel-cap.

The tablets or capsules can be coated, if desired, with a suitable enteric coating so as to assist in passing through the stomach without degradation. The pH in the stomach is of the order of 1 to 3 but it increases in the small intestine and the colon to attain values close to 7 (Hovgaard L. et al. (1996) Current Applications of Polysaccharides in Colon Targeting, Critical Reviews in Therapeutic Drug Carrier Systems, 13, 185). The drug delivery devices, in the form of tablets, gelatine capsules, spheroids and the like, can reach the colon, without being exposed to these variations in pH, by coating them with a pH-dependent polymer, insoluble to acidic pH but soluble in neutral or alkaline pH (Kinget et al. op. cit.). The polymers most current used for this purpose are derivatives of methacrylic acid, Eudragit® L and S (Ashford M. et al. (1993), An in vivo investigation into the suitability of pH-dependent polymers for colonic targeting, International Journal of Pharmaceutics, 95, 193 and 95, 241; and David A. et al. (1997) Acrylic polymers for colon-specific drug delivery, S.T.P. Pharma Sciences, 7, 546).

The drug delivery devices are administered in an effective amount suitable to provide some degree of treatment or prevention of the disorders for which the compounds are administered. The effective amounts of those compounds are typically below the threshold concentration required to elicit any appreciable side effects. The compounds can be administered in a therapeutic window in which certain the disorders are treated and certain side effects are avoided. Ideally, the effective dose of the compounds described herein is sufficient to provide the desired effects in the colon but is insufficient (i.e., is not at a high enough level) to provide undesirable side effects elsewhere in the body.

Most preferably, effective doses are at very low concentrations, where maximal effects are observed to occur, with a minimum of side effects, and this is maximized by targeted colonic delivery of the active agents. Typically, the effective dose of such compounds generally requires administering the compound in an amount less than 100 mg/kg of patient weight, often less than about 0.1 mg/kg patient weight and usually, but frequently, between about 1 mg to less than 10 mg/kg of patient weight. The foregoing effective doses typically represent that amount administered as a single dose, or as one or more doses administered over a 24-hour period.

### IV. Methods of Treatment Using the Drug Delivery Devices

The drug delivery devices can be used to treat those types of conditions and disorders for which colonic delivery is appropriate. In one embodiment, the disorders are those that result from exposure of the colon to antibiotics, such as diarrhea. In this embodiment, the drug delivery devices include agents which inactivate antibiotics, and the devices can be administered in a therapeutically effective dosage to a patient who has been, is being, or will be administered an antibiotic.

In another embodiment, the drug delivery devices are administered to a patient who suffers from colon cancer. In this embodiment, the drug delivery devices include one or more antitumor agents, and the devices are administered in a therapeutically effective dosage to a patient who is suffering from colon cancer. Alternatively, the cancer can be present at another location in the body, and the drug delivery devices can be used to by-pass the stomach and its concomitant degradation of certain antitumor agents, so as to avoid the need to use intramuscular or intravenous administration of these agents.

In another embodiment, the drug delivery devices are administered to a patient who suffers from a colonic disorder such as Chrohn's disease, ulcerative colitis, irritable bowel syndrome, diarrhea, or constipation. In this embodiment, the drug delivery devices include agents which treat or prevent these disorders, and the devices can be administered in a therapeutically effective dosage to a patient who is suffering from such a disorder.

In still another embodiment, the drug delivery devices are used to administer peptide or protein-based active agents, such as insulin, antibodies, and the like, or oligonucleotide-based therapeutics, such as antisense therapy, so that the agents pass through the stomach without being digested. In this embodiment, the drug delivery devices include these protein/peptide/oligonucleotide-based agents, and the devices can be administered in a therapeutically effective dosage to a patient in need of treatment with these agents, without the need to administer these agents via subcutaneous or intravenous injection.

In a further embodiment, the drug delivery devices are used to administer diagnostic agents to the colon. In this embodiment, the drug delivery devices include diagnostic agents, such as imaging contrast agents, and the devices are administered in a diagnostically effective dosage to a patient who will be subjected to a diagnostic assay for diagnosis of a colonic disorder.

The present invention will be further understood with reference to the following non-limiting examples.

### EXAMPLE 1

### Preparation of Galenic Forms

An aqueous solution of pectin at 6% (OF 400 or OG175C Unipectint by Degussa) was introduced dropwise to a solution of calcium chloride at 6% (m/v). The solution of pectin was introduced to the solution of calcium chloride via Tygon piping connected to a peristaltic pump (Microperpexe LKB Bromma). The solution was passed through a needle of 0.8 mm in diameter (21G, Nedus Terumo) to form drops of pectin which gelled instantly on contact with the calcium chloride (40 ml) and yielded beads of calcium pectinate. The beads were kept in the calcium chloride, with slow stirring, for 20 minutes.

The white beads not containing active ingredient (β-lactamases) were obtained starting out from a solution of amidated (OG 175C) or non-amidated (OF 400) pectin at 6%. For preparation of loaded beads the active ingredient (β-lactamases, penicillinases of type A extracted from Bacillus cereus by Sigma) was mixed in with the solution of pectin in a ratio of 3% (Vpa/Vpectin).

The resulting beads of calcium pectinate were then recovered by filtration, rinsed in distilled water, placed on a Petri dish and dried by kiln at 37° C. for 2 hours.

For reticulation in polyethylenimine the undried beads, recovered from the solution of CaCl₂ by filtration, were introduced to an aqueous solution of polyethylenimine (PEI) at 1% and were kept there for 20 min with gentle stirring.

The beads prepared from the non-amidated pectin OF 400 contained from 1 to 2.5 UI/beads and the beads prepared from amidated pectin OG175C contained from 1 to 5 UI/beads.

### EXAMPLE 2

### Stability of Beads

1. Operational method.

The beads were prepared according to Example 1 with or without the reticulation stage; the duration of reticulation in PEI was 20 minutes in solutions of concentrations ranging from 0.6 to 1%.

The beads were placed either in phosphate buffer (PBS O,01M, pH 7.4), or in media simulating digestive juices (gastric and intestinal USP XXIV) and the disaggregation time was observed.

### 2. Results.

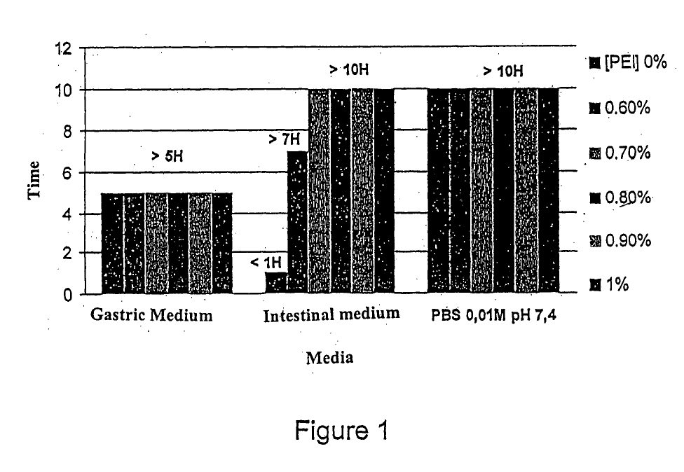

These are given in FIG. 1.

The beads reticulated or not were stable in the PBS and in the gastric medium. However, the non-reticulated beads were unstable in the intestinal medium, whereas the beads according to the invention were stable for over 7 hours.

### EXAMPLE 3

### Morphological Characteristics of Beads

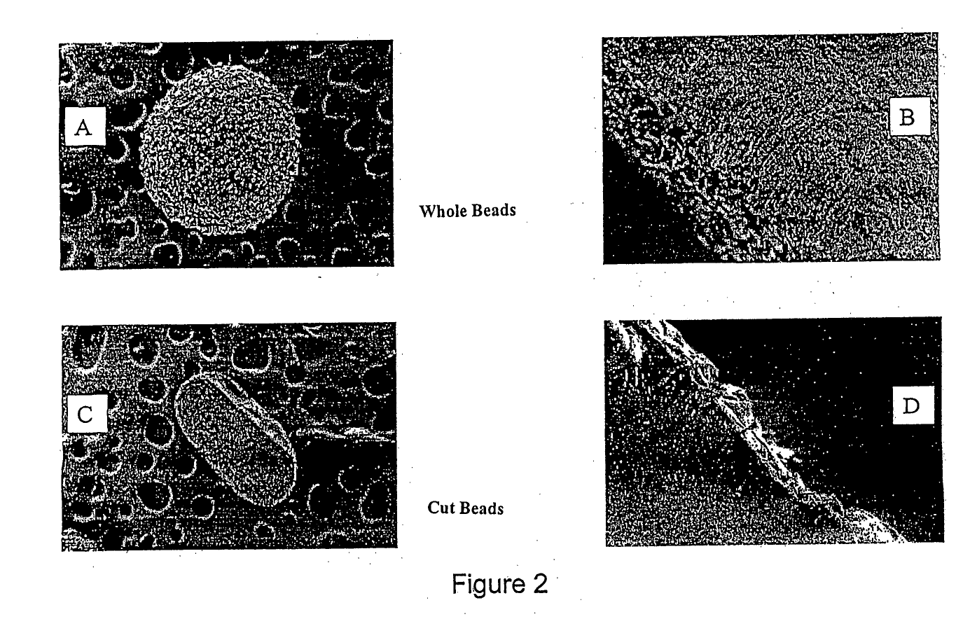

They are illustrated in FIGS. 2A to 2D.

The cuts show that the centre of the beads was full and dense. The surface shell corresponds to the PEI. The interior and exterior have different structures.

### EXAMPLE 4

### Release Kinetics In Vitro

1. Operational method.

Beads reticulated with two different concentrations of PEI (0.6 and 0.7%) were prepared according to Example 1 from amidated pectin and containing 5 UI/bead. They were left for 5 hours in intestinal medium USP XXIV at pH 6.8, then introduced to synthetic colonic medium at pH 6 including pectinolytic enzymes (Pectinex Ultra SPL).

The residual β-lactamase activity in the beads was measured over time by spectrophotometry in the presence of nitrocephine.

2. Results.

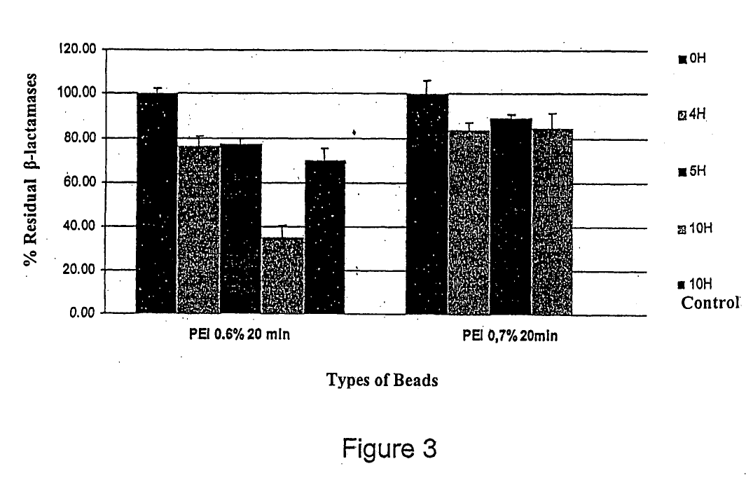

They are illustrated in FIG. 3.

After 5 hours of incubation beads in intestinal medium (T_{5H}) less 25% of β-lactamase activity which they contain was released. The release becomes important in colonic medium under the action of pectinolytic enzymes (Tien), for the reticulated beads with 0.6% PEI, while the sample without pectinolytic enzymes (T_{10H} control) had no significant modifications of β-lactamase activity. On the contrary, the beads reticulated with 0.7% PEI did not have their activity diminish after 5H in colonic medium.

Thus the concentration of PEI modifies the resistance of beads and plays on the release time of the active ingredients in colonic medium.

### EXAMPLE 5

### Release Kinetics In Vivo

1. Operational method.

This assay was performed on male mice CD1. The beads contain 4 UI/bead.

Gels containing 10 beads were administered per os to the mice. The stools were recovered at time periods of 0, 2H, 3H, 4H, 5H, 6H, 7H and 8H and the dosage of β-lactamases in these stools was realized (assay conducted on 5 animals for each time). In addition, one mouse was sacrificed at times of 30 min, 2H and 4H so as to recover the beads in its digestive tract and observe their morphological modifications by scanning electron microscopy.

2. Results.

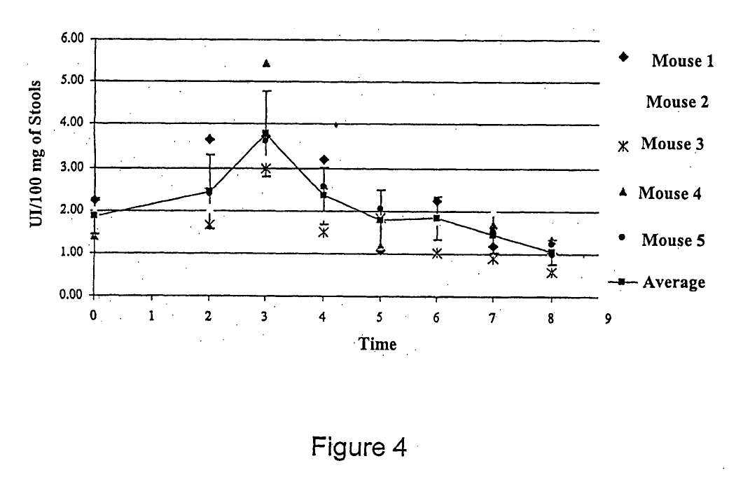

These are illustrated in FIGS. 4 to 7.

The beads arrived intact in the colon after around 3 hours' transit.

The rate of β-lactamases released directly in the stools of mice gathered at different times after absorption of the beads orally shows that the basic β-lactamase activity is low at the outset. Two to 4 hours after administration there was a clear increase in this activity, corresponding to the transit of the beads (FIG. 4).

The photos taken by scanning electron microscopy show the integrity of the bead at different places of the digestive tract.

The structure is slightly fragile in the small intestine and the inside was completely destroyed at colonic level where the beads appeared carriers of a cavity.

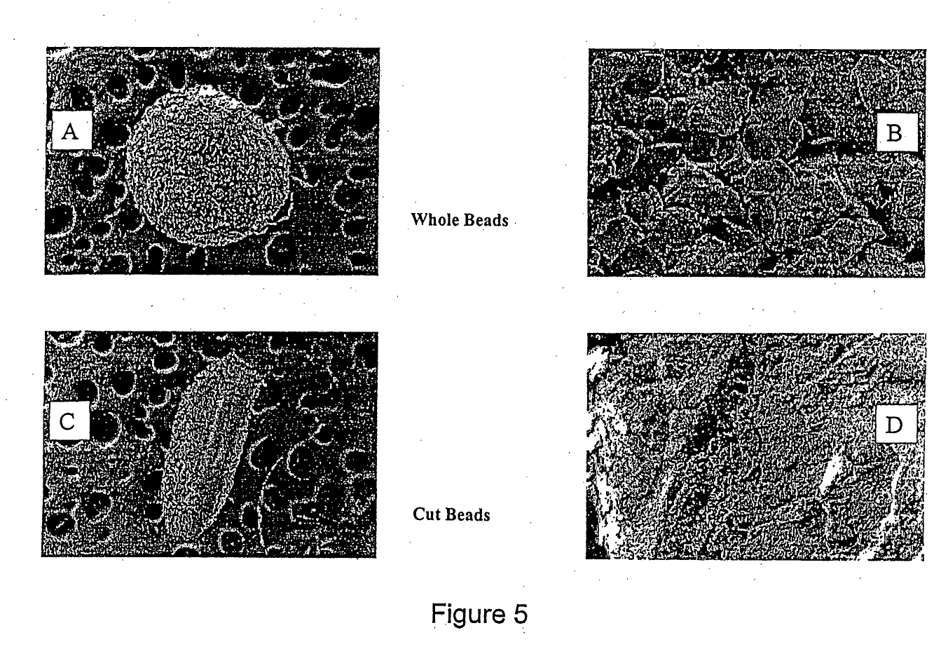

As illustrated in FIG. 5 the particles, having stayed in the stomach, looked very similar to those which had not undergone any treatment (FIG. 2). In effect the surface had the same rugged and irregular look (FIGS. 5A and 5B), owing to the presence of polyethylenimine, and the cross-section of the beads appeared uniform and dense (FIGS. 5C and 5D).

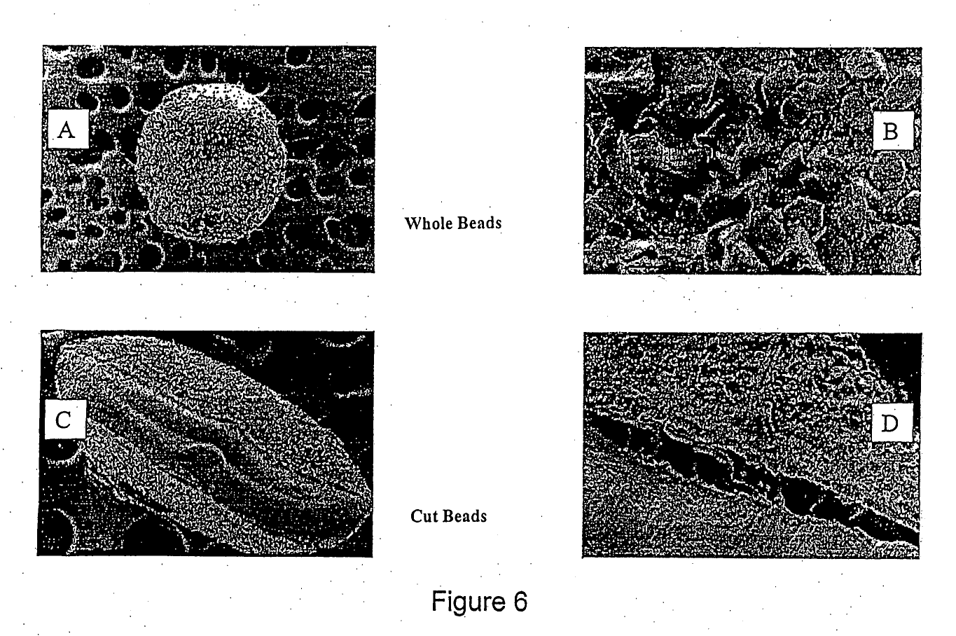

At the end of 2 h slight deformation of the beads became apparent (FIG. 6A), but the particles still had the same surface appearance (FIG. 6B) and a dense cross-section (FIG. 6C), even though they were made a little fragile by their stay in the small intestine (FIG. 6D).

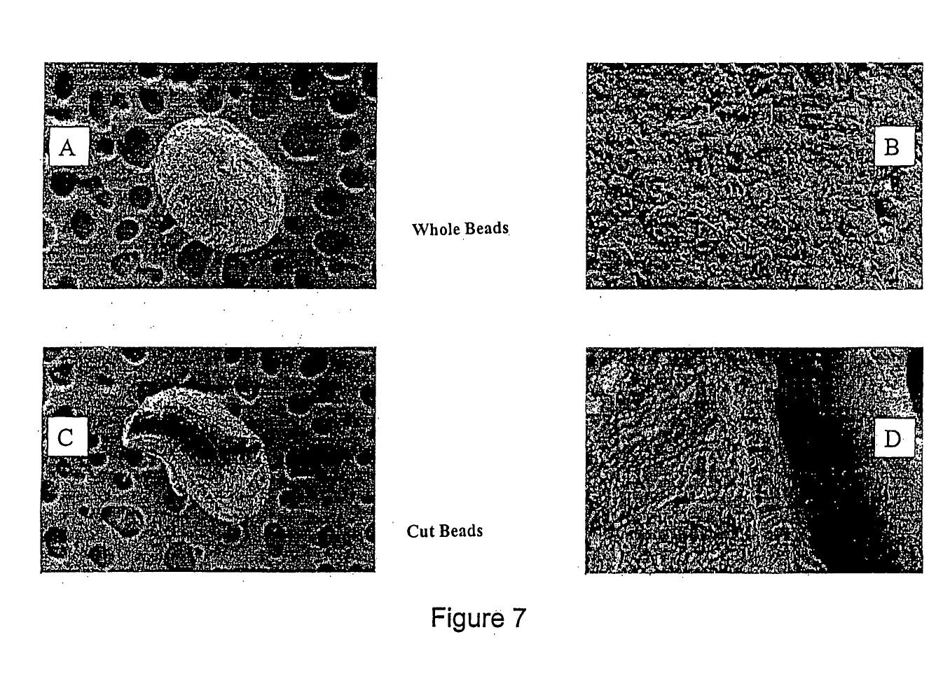

On completion of transit, that is, 4 h after administration, the beads were in the colon; the external appearance of the particles was unchanged (FIG. 7A) with the same surface irregularities due to the polyethylenimine (FIG. 7B). Yet the cross-section of the beads was hollow (FIGS. 7C and 7D), due to the fact of degradation of the central network of calcium pectinate by the colonic pectinolytic enzymes. Finally, only the external shell formed by the polyethylenimine remained.

### EXAMPLE 6

### Encapsulation of Erythromycin Esterase

6.1 Production of a Soluble Fraction Containing Erythromycin Esterase

6.1.1. Operational Method

The culture was made from the strain of E. coli C600 pIP1100 from the Pasteur Institute. The culture conditions were the following: inoculation of the Mueller-Hinton medium at 0.5% from a preculture of about 20 h, culture volumes of 200 or 400 mL in Erlenmeyer, fixed agitation at 150 rpm, temperature of 37° C.

A GOTS test helped establish that the strain produced much erythromycin esterase.

3.6 L of culture of E. coli C600 pIP1100 were concentrated according to the following protocol:

Centrifuging for 15 min at 3400 g

Recovery of cap in potassium phosphate buffer 5 mM, pH 7.5, final volume 70 mL

Second centrifuging of supernatant for 15 min at 3400 g

Recovery of cap in 20 mL of potassium phosphate buffer 5 mM, pH 7.5

Reuniting of caps of the 2 centrifuges (around 100 mL)

Washing of caps and centrifuging (10 min at 12,000 g)

Second centrifuging of supernatant (10 min 12,000 g)

Final volume of caps recovered in the potassium phosphate buffer: 100 mL.

The erythromycin esterase was an intracellular enzyme. This is why its solubilization required the cells to be broken. This operation was carried out by ultrasonic extraction of centrifuging caps recovered in the potassium phosphate buffer 5 mM, pH 7.5 according to the protocol described hereinbelow.

Addition of 1% TritonX100 (v/v)

Cooling to 5° C.

Phonolysis 7 cycles of 1 min, initial temperature 5° C., maximal temperature 15° C., power: 100% (500W, 20 kHz); temperature taken to 5° C. after each cycle

Centrifuging for 10 min at 12,000 g

Recovery of cap in 10 mL of potassium phosphate buffer 5 mM, pH 7.5

Recovery and congealing of the supernatant (91 mL)=solution A.

The erythromycin esterase activity was evaluated by the microbiological dosage in the supernatant and in the insoluble substances (cellular debris) according to techniques known to the expert.

6.1.2. Results

The results are presented in Table 2.

TABLE 2

Diameter of inhibition (mm)

| Sample | T0 | T30 | T60 | T120 |
|---|---|---|---|---|
| Supernatant | 31 | 25 | 18 | — |
| after | 21 | — | 18 | 14 |
| ultrasound | | | | |
| Cap after | 30 | 28 | 24 | — |
| ultrasound | 22.5 | — | 19.5 | 19 |

The erythromycin esterase activity was evaluated from the diameter of inhibition.

The latter was 2 U/mL for the phonolysis supernatant and 1.5 U/mL for the phonolysis cap (1 Unit (U)=1 μg of erythromycin degraded per min).

The balance of recovery of the erythromycin esterase activity is presented in Table 3 hereinbelow.

TABLE 3

| | Estimated activity | | Total estimated |
|---|---|---|---|
| Sample | (U/mL) | Volume (mL) | activity (U) |
| Supernatant after | 2.0 | 92 | 184 |
| ultrasound | | | |
| Cap after | 1.5 | 10 | 15 |
| ultrasound | | | |

The results clearly show that the essential element in the erythromycin esterase activity present has been solubilized in the phonolysis medium.

6.2 Encapsulation of Erythromycin Esterase

6.2.1. Operational Method

Encapsulation was achieved from the non-purified soluble fraction obtained after breaking the cells (solution A) according to the following protocol.

Solubilization of 0.5 g of pectin in 10 mL solution A to obtain a final concentration of pectin of 5% (solution B). The pectin was added very progressively with magnetic stirring so as not to cause too many abrupt variations in pH. The pH was maintained in the region of 7 by addition of a few drops of soda 1 M.

Dispersion of the solution of pectin (solution B) dropwise by means of a peristaltic pump to 40 mL of CaCl₂ at 6%. The beads thus formed were kept in the CaCl₂ for 20 min, recovered by Büchner filtration then rinsed in demineralized water.

Reticulation of the beads by bath in a solution of PEI at 0.6% for 20 min with magnetic stirring.

Recovery of the reticulated beads by filtration.

The beads were dried at ambient temperature (20° C.). 567 beads were prepared in total with 6.1 mL of pectin/solution A mixture, for an activity of 12.2 U.

The dried beads were disaggregated in a buffer HEPES/NaCl/EDTA 1%.

6.2.2. Results

The erythromycin esterase activity present in the initial solution of pectin and that released in the disaggregation medium were dosed according to the same protocol as previously.

The results of the microbiological dosage are presented in Tables 4 and 5.

TABLE 4

| Sample | Average inhibition diameter (mm) |
|---|---|
| Pectin/Solution A | 23 |
| (solution B) -T0 | |
| Pectin/Solution A -T3 h | 19 |
| Disaggregated beads - T0 | 24 |
| Disaggregated beads - T3 h | 18 |

TABLE 5

| Sample | Estimated activity |
|---|---|
| Pectin (Solution B) | 2.4 |
| Disaggregated beads | 2.2 |

The results show that the activity measured in the presence of pectin (solution B) is 2.4 U, while the theoretical activity present should be around 12 U (6.1 mL at 2 U/mL, according to the dosage of erythromycin esterase in the phonolysis supernatant (Table 3).

The dosage of enzymatic activity of beads after disaggregation had been estimated at 2.2 U; it represented 90% of the initial activity introduced to the beads.

These results help confirm unambiguously the presence of erythromycin esterase activity in the final fraction after encapsulation of the enzyme and disaggregation of the beads.

### EXAMPLE 7

### Encapsulation of DNA in the Calcium Pectinate Beads

7.1 Preparation of DNA

The active ingredient encapsulated here was a plasmid radiomarked with Phosphore 33. The radiomarking was done by means of the Nick Translation Kit N5500 from Amersham Biosciences according to the protocol described by the supplier.

7.2 Encapsulation

7.2.1. Operational Method

The encapsulated DNA was either in free form, or complexed with cationic lipids (Lipoplexe) or a cationic polymer (Polyplexe) according to the operational method described in Example 1.

For free DNA, around 5 μg of DNA radiomarked in solution in 750 μL of MilliQ water were introduced to 0.75 g of a pectin solution, amidated or not, at 10% so as to obtain a final concentration of pectin of 5%. In the case of the lipoplexes, 375 μL of an aqueous solution of radiomarked DNA were mixed with 375 μL of a suspension of cationic liposomes (N/P ratio of 10). The 750 μL of resulting lipoplexes were then mixed with 0.75 g of solution of pectin at 10% so as to obtain a final concentration of pectin of 5%.

In the case of polyplexes, 375 μL of an aqueous solution of radiomarked DNA was mixed with 375 μL of an aqueous solution of PEI 4 mM. 375 μL of the suspension of polyplexes thus obtained were then mixed with 0.75 g of pectin solution at 10% to provide a final concentration of pectin of 5%.

The beads of calcium pectinate encapsulating the free or complex DNA were then prepared from solutions obtained hereinabove according to the method described in Example 1.

The concentration of calcium chloride utilized here was 5% and that of PEI for reticulation was 0.6%.

7.2.2. Results

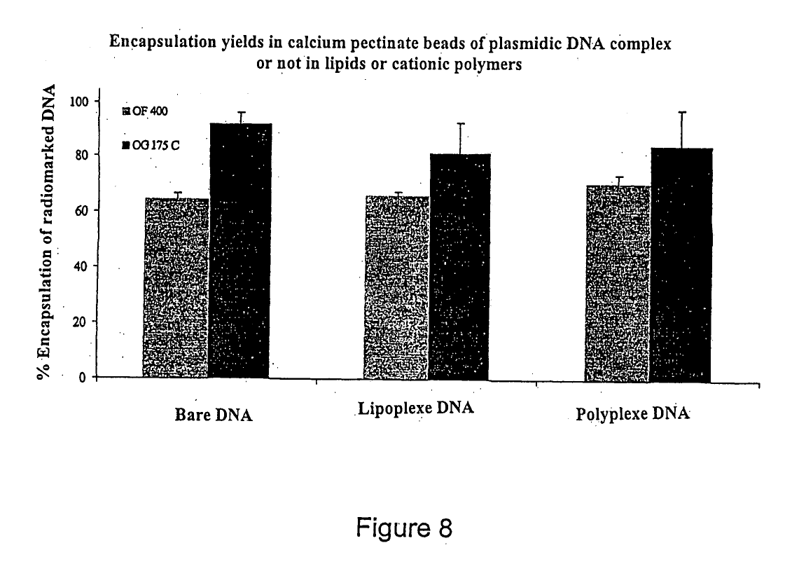

The results are illustrated in FIG. 8 which shows the encapsulation yields of a plasmid DNA in amidated or non-amidated pectin beads.

The encapsulated DNA was either in free form, or complexed in cationic lipids (Lipoplexe) or a cationic polymer (Polyplexe).

The encapsulation yields of DNA varied between 60 and 90% according to the type of pectin used. They were generally more significant with amidated pectin. Complexing with lipids or a cationic polymer did not cause significant modifications to these yields, which remained relatively high.

### EXAMPLE 8

### Preparation of Polyethyleneimine-Reticulated Pectin Beads

The beads were prepared by the procedure described in the PCT WO 2004/016248 by Bourgeois et al.

They were then coated/reticulated with several coating/reticulating agents. Stability was determined by observing time for disintegration in simulated intestinal medium. In those embodiments where the beads showed a stability greater than 5 hours, the residual activity was measured. The stability of beads coated/reticulated with different cross-linking solutions was measured in simulated intestinal medium (SIM). The results are shown below in Table 6.

TABLE 6

| Coating solution | Stability | | Remaining activity | |
|---|---|---|---|---|
| Nude beads | 2 | hours | | |
| | | (1, 3, 6, 7, 8) | | |
| PEI 0.8% | >8 | hours (2) | 82.7% | after 5 h (2) |
| | >5 | hours (1) | 98.9% | after 5 h (1) |
| | 4 | hours (7) | | |
| Chitosan 1% high Mw | 3.5 | hours | 19.8% | after 2 h (2) |
| Chitosan low M_{w}1% | 5+ | hours (3) | 85.9% | after 2 h |
| | <3 | hours (4) | 22% | after 5.5 h (3) |
| | 2 | hours (7) | | |
| Chitosan low M_{w} 1.25% | <3 | hours (4) | | |
| Chitosan low M_{w} 1.5% | <3 | hours (4) | | |
| Chitosan low M_{w} 1.75% | <3 | hours (4) | | |
| Chitosan low M_{w} 2% | <3 | hours (4) | | |
| Chitosan 0.1% + | <3 | hours (5) | Prepared by the Munjeri | |
| 1.8% CaCl₂ | | | method | |
| Chitosan 1% + | <3 | hours (5) | Munjeri et al. ((1997) | |
| 1% CaCl₂ | | | Hydrogel beads based on | |
| Chitosan 0.1% + | <3 | hours (5) | amidated pectins for colon- | |
| 5.3% CaCl₂ | | | specific drug delivery: the | |
| Chitosan 1% + | <3 | hours (5) | role of chitosan in | |
| 3% CaCl₂ | | | modifying drug release, | |
| | | | Journal of Controlled | |
| | | | Release, 46, 273) | |
| Eudragit 13% | 2 | hours (6) | Rude surface | |
| | 2 | hours (7) | Solutions in ethanol/water | |
| Eudragit 3.25% | 2 | hours (7) | | |
| Eudragit 6.5% | 2 | hours (7) | | |
| Hydroxyethylcellulose | 2 | hours (6, 7) | | |
| ethoxylate, quaternized | | | | |
| 0.1% | | | | |
| PEI 0.8% + chitosan 1% | 2 | hours (8) | | |

(1) Stability of cross-linked calcium pectinate beads in simulated intestinal media (SIM)
(2) Intestinal stability of calcium pectinate beads cross-linked with chitosan compared to beads cross-linked with PEI
(3) Intestinal stability of penicillinase loaded calcium pectinate beads cross-linked with low M_{w} chitosan
(4) Intestinal stability and degradation of pectine beads cross-linked with chitosan at different concentrations
(5) Stability of pectin beads cross-linked with chitosan prepared by the Munjeri procedure
(6) Stability testing of calcium pectinate beads cross-linked with different polymers to improve and compare stability
(7) Intestinal stability of calcium pectinate cross-linked with Eudragit at different concentrations
(8) PEI and chitosan mix for cross-linking

The data show that polyethyleneimine is able to stabilize the pectin beads significantly better than the other polymers which were evaluated.

Each document referred to herein is hereby incorporated by reference in its entirety for all purposes.

Having hereby disclosed the subject matter of the present invention, it should be apparent that many modifications, substitutions, and variations of the present invention are possible in light thereof. It is to be understood that the present invention can be practiced other than as specifically described. Such modifications, substitutions and variations are intended to be within the scope of the present application.

1. Oral drug delivery devices for colonic release of active ingredients, comprising:
a) an active agent capable of inactivating an antibiotic, and
b) a drug delivery device comprising pectin beads, where the pectin is crosslinked with calcium ions, and reticulated with polyethyleneimine.

2. The drug delivery device of claim 1, wherein the amount of polyethyleneimine is sufficient to allow a substantial portion of the pectin beads to pass through the gastrointestinal tract to the colon without releasing the active agent, and is also sufficient such that the pectin beads are sufficiently degraded in the colon to release an effective amount of the active agent.

3. The drug delivery device of claim 1, wherein the active agent is an enzyme capable of inactivating beta-lactam antibiotics.

4. The drug delivery device of claim 1, wherein the active agent is an enzyme capable of inactivating macrolide or quinolone antibiotics.

5. The drug delivery device of claim 4, wherein the enzyme capable of inactivating macrolides is erythromycin esterase.

6. The drug delivery device of claim 1, wherein the polyethyleneimine has a molecular weight between 20,000 and 50,000 Daltons.

7. The drug delivery device of claim 1, wherein the pectin is amidated pectin.

8. The drug delivery device of claim 1, wherein the device is prepared from a 4-10% (m/v) pectin solution, a 2-10% (m/v) calcium chloride solution, and a 0.5-2% (m/v) polyethylenimine solution.

9. A method for treating or preventing adverse effects of an antibiotic to the intestinal flora, comprising administering the drug delivery device of claim 1 to a patient, either before, during, or after administration of the antibiotic.

10. A process for preparing an oral drug delivery device for delivery of an active agent that inactivates an antibiotic to the colon, comprising:
a) adding an aqueous pectin solution containing a dissolved, dispersed or suspended active agent, where the agent inactivates an antibiotic, to an aqueous solution of a divalent cationic salt, so as to obtain beads of pectin in the form of a cationic salt including the active agent, and
reticulating the resulting beads by introducing them to an aqueous solution of polyethyleneimine.

11. The process of claim 10, wherein the cationic salt is a calcium ion.

12. The process of claim 10, wherein the polyethyleneimine has a molecular weight between 10,000 and 100,000 Daltons.

13. The process of claim 10, wherein the polyethyleneimine has a molecular weight between 20,000 and 50,000 Daltons.

14. The process of claim 10, wherein the amount of polyethyleneimine is sufficient to allow a substantial portion of the pectin beads to pass through the gastrointestinal tract to the colon without releasing the active agent, and is also sufficient such that the pectin beads are sufficiently degraded in the colon to release an effective amount of the active agent.

15. The process of claim 10, wherein the active agent is an enzyme capable of inactivating beta-lactam, macrolide or quinolone antibiotics.

16. The process of claim 15, wherein the enzyme capable of inactivating macrolides is erythromycin esterase.

17. The process of claim 10, wherein the active agent is a beta-lactamase.

18. Oral drug delivery devices for colonic release of active ingredients, comprising:
a) an active agent capable of treating disorders of the colon, and
b) a drug delivery device comprising pectin beads, where the pectin is crosslinked with calcium ions, and reticulated with polyethylene imine.

19. The oral drug delivery device of claim 18, wherein the amount of polyethyleneimine is sufficient to allow a substantial portion of the pectin beads to pass through the gastrointestinal tract to the colon without releasing the active agent, and is also sufficient such that the pectin beads are sufficiently degraded in the colon to release an effective amount of the active agent.

20. The drug delivery device of claim 19, wherein the disorder is Crohn's disease or ulcerative colitis, and the active agent is selected from the group consisting of minosalicylates, drugs that contain 5-aminosalicyclic acid (5-ASA), corticosteroids, immunomodulators, cyclosporine A, TNF alpha, thiazoldinediones and glitazones.

21. A method of treating Chrohn's disease or ulcerative colitis, comprising administering an effective amount of the drug delivery device of claim 20 to a patient in need of treatment thereof.

22. The drug delivery device of claim 18, wherein the disorder is colon cancer, and the active agent is selected from the group consisting of anti-proliferative agents, agents for DNA modification or repair, DNA synthesis inhibitors, DNA/RNA transcription regulators, enzyme activators, enzyme inhibitors, gene regulators, HSP-90 inhibitors, microtubule inhibitors, agents for phototherapy, and therapy adjuncts.

23. A method of treating colon cancer, comprising administering an effective amount of the drug delivery device of claim 22 to a patient in need of treatment thereof.

24. The drug delivery device of claim 18, wherein the disorder is irritable bowel syndrome or constipation, and the active agent is selected from the group consisting of stimulant laxatives, osmotic laxatives, stool softeners, bulking agents, Zelnorm (tegaserod), and anticholinergic medications.

25. A method of treating irritable bowel syndrome or constipation, comprising administering an effective amount of the drug delivery device of claim 24 to a patient in need of treatment thereof.

26. The drug delivery device of claim 18, wherein the device is used as a diagnostic agent, and the encapsulated agent is a diagnostic agent.

27. The drug delivery device of claim 26, wherein the diagnostic agent is selected from the group consisting of radiolabeled compounds, radioopaque compounds, and gases.

28. A method of diagnosing a disorder in the colon, comprising:
a) administering an effective amount of the drug delivery device of claim 27 to a patient in need of diagnosis thereof, and
b) detecting the diagnostic agent.
